(19) United States
(12) Patent Application Publication
(10) Pub. No.: US 2022/0290238 A1
(43) Pub. Date: Sep. 15, 2022

(54) BLOOD GENE BIOMARKERS TO DIAGNOSE AND PREDICT ACUTE REJECTION IN LIVER TRANSPLANT RECIPIENTS

(71) Applicant: NORTHWESTERN UNIVERSITY, Evanston, IL (US)

(72) Inventors: Michael M. Abecassis, Highland Park, IL (US); Josh Levitsky, Evanston, IL (US)

(21) Appl. No.: 17/637,410

(22) PCT Filed: Aug. 21, 2020
(86) PCT No.: PCT/US20/47481

§ 371 Date: Feb. 22, 2022

Related U.S. Application Data
(60) Provisional application No. 62/891,094, filed on Aug. 23, 2019.

(51) Int. Cl. C12Q 1/6883

(52) U.S. Cl. CPC C12Q 1/6883; C12Q 2600/106; C12Q 2600/158; C12Q 2600/118

(57) ABSTRACT

The present disclosure is directed to materials and methods for predicting and diagnosing acute rejection in liver transplant recipients. Methods of the disclosure are useful, in various embodiments, for adjusting or initiating therapies (e.g., immunosuppressive (IS) therapy) in patients who would benefit therefrom.

### CROSS-REFERENCE TO RELATED APPLICATIONS

This application claims the priority benefit under 35 U.S.C. § 119(e) of U.S. Provisional Patent Application No. 62/891,094, filed Aug. 23, 2019, which is incorporated herein by reference in its entirety.

### STATEMENT OF GOVERNMENT INTEREST

This invention was made with government support under grant number U01 AI084146 awarded by the National Institutes of Health (NIH). The government has certain rights in the invention.

### FIELD OF THE INVENTION

The present disclosure is directed to materials and methods for predicting and diagnosing acute rejection in liver transplant recipients. Methods of the disclosure are useful, in various embodiments, for adjusting or initiating therapies (e.g., immunosuppressive (IS) therapy) in patients who would benefit therefrom.

### BACKGROUND

The advent of calcineurin inhibitors (CNI) has resulted in more acceptable rates of acute rejection (AR) following liver transplantation (LT), and the shift from cyclosporine to tacrolimus-based immunosuppression (IS) regimens significantly reduced the incidence of chronic rejection (1). However, LT recipients are at high risk for IS complications such as chronic kidney disease (CKD), malignancy, cardiovascular disease, metabolic bone disease, and other issues related to adherence and cost (2, 3). In response to observations that some patients may not require sustained exposure to long-term IS, studies have shown that IS minimization, particularly CNI-sparing strategies, or even full IS withdrawal attempts may be feasible and potentially beneficial to long-term outcomes (3-6). However, strategies to withdraw CNI therapy early after LT, where the benefit on renal and metabolic parameters may be the greatest, have been limited by the development of AR during these interventions, leaving most patients on long term therapy (7-9). In addition, approaches involving complete IS withdrawal (e.g. tolerance) require serial and invasive liver biopsies to confirm that the graft is free of rejection, even in patients with normal liver function tests (LFTs) (9-11).

Despite a significant and longstanding interest in developing immune monitoring strategies, there are currently no available assays other than IS drug levels, LFTs or biopsies which are either suboptimal or impractical for assessing individual immunosuppression requirements (12-14).

### SUMMARY

Multiple genomic biomarkers have been proposed to differentiate diagnostically between causes of graft dysfunction (15-29), but these have not been subjected to robust validation or been tested as predictive biomarkers, preceding and following treatment of AR. Studies described and disclosed herein were designed to discover and validate molecular biomarkers for a number of clinical phenotypes following LT. The focus of the studies was to evaluate the performance of a gene expression biomarker that correlates with the presence or absence of rejection in the peripheral blood of patients following LT. The present disclosure provides, in various aspects, the ability to predict rejection and healthy transplant following liver transplantation, to more objectively guide immunosuppressive therapy management. The ability to predict rejection is essential for a number of reasons including minimizing the occurrence of both acute rejection and immunosuppressive complications in this population.

Accordingly, in some aspects the disclosure provides a method of adjusting or initiating immunosuppressive therapy in a subject who has undergone a liver transplant, comprising: a) determining an expression level of one or more genes listed in Table 3 using nucleic acid obtained from the subject, wherein the one or more genes comprises NHS actin remodeling regulator (NHS), endothelial PAS domain protein 1 (EPAS1), complement component 4 binding protein alpha (C4BPA), lymphocyte cytosolic protein 2 (LCP2), deleted in lymphocytic leukemia 2 (DLEU2), NHS like 1 (NHSL1), required for meiotic nuclear division 5 homolog A (RMND5A), lymphocyte activating 3 (LAG3), chitinase 3 like 2 (CHI3L2), gelsolin (GSN), ASAP1 intronic transcript 2 (ASAP1-IT2), NLR family apoptosis inhibitory protein (NAIP), geminin DNA replication inhibitor (GMNN), C-type lectin domain family 4 member E (CLEC4E), X inactive specific transcript (XIST), long intergenic non-protein coding RNA 877 (LINC00877), A-kinase anchoring protein 12 (AKAP12), ATPase family AAA domain containing 2 (ATAD2), or a combination of any of the foregoing; b) applying the expression level of the genes determined in step (a) to a trained algorithm to classify the subject as (i) undergoing acute rejection (AR) or at risk of undergoing AR or (ii) not undergoing AR or not at risk of undergoing AR; and c) adjusting or initiating the immunosuppressive therapy in the subject based on classification of the subject in step (b). In some embodiments, step (a) further comprises determining an expression level of stathmin 1 (STMN1), protein phosphatase 1 regulatory subunit 12B (PPP1R12B), mesoderm specific transcript (MEST), ribonucleotide reductase regulatory subunit M2 (RRM2), senataxin (SETX), thymidylate synthetase (TYMS), GATA binding protein 2 (GATA2), KIAA1324, or a combination of any of the foregoing. In some aspects, the disclosure provides a method of adjusting or initiating immunosuppressive therapy in a subject who has undergone a liver transplant, comprising: a) determining an expression level of one or more genes listed in Table 3 using nucleic acid obtained from the subject, wherein the one or more genes is gene number 3, 6, 7, 8, 10, 11, 13, 14, 15, 16, 18, 19, 20, 22, 23, 24, 25, 26, 27, 28, 29, 30, 32, 34, 35, or 36; b) applying the expression level of the genes determined in step (a) to a trained algorithm to classify the subject as (i) undergoing acute rejection (AR) or at risk of undergoing AR or (ii) not undergoing AR or not at risk of undergoing AR; and c) adjusting or initiating the immunosuppressive therapy in the subject based on classification of the subject in step (b). In some embodiments, step (a) further comprises determining an expression level of one or more additional gene listed in Table 3, wherein the one or more additional genes is gene number 1, 2, 4, 5, 9, 12, 17, 21, 31, or 33. In some embodiments, step (a) comprises determining the expression level of 5, 10, 15, 20, 25, 30, 35, or each of the genes listed in Table 3. In some aspects, the disclosure provides a method of adjusting or initiating immunosuppressive therapy in a subject who has undergone a liver transplant, comprising: a) determining an expression level of each of the genes listed in Table 3 using nucleic acid obtained from the subject; b) applying the expression level of the genes determined in step (a) to a trained algorithm to classify the subject as (i) undergoing acute rejection (AR) or at risk of undergoing AR or (ii) not undergoing AR or not at risk of undergoing AR; and c) adjusting or initiating the immunosuppressive therapy in the subject based on classification of the subject in step (b). In some embodiments, the expression level is determined by hybridization to an array or RNA sequencing. In some embodiments, the subject has normal liver function at the time the subject is classified. In further embodiments, the normal liver function is determined by a liver function test (LFT) in which total bilirubin (TB) is less than 1.5 mg/dL, direct bilirubin is less than 0.5 mg/dL, alkaline phosphatase (AP) is less than 200 U/L, and alanine transaminase (ALT) is less than 60 U/L (males) or less than 36 U/L (females). In some embodiments, a method of the disclosure further comprises comparing the expression level of each of the genes to a reference expression level that is associated with presence or absence of acute rejection. In some embodiments, the classifying of the subject has a negative predictive value of at least 75%, at least 80%, at least 85%, at least 90%, at least 95%, or at least 99%. In some embodiments, the immunosuppressive therapy or adjusted immunosuppressive therapy comprises administering a calcineurin inhibitor, a mycophenolic acid derivative, an mTOR inhibitor, prednisone, azathioprine, or a combination thereof. In some embodiments, the trained algorithm comprises a random-forest-based algorithm. In some embodiments, the adjusting or initiation of the immunosuppressive therapy comprises increasing dose or frequency of the immunosuppressive therapy in the subject undergoing or at risk of undergoing AR. In some embodiments, the subject is not receiving immunosuppressive therapy at the time the subject is classified. In some embodiments, the adjusting of the immunosuppressive therapy comprises decreasing dose or frequency of the immunosuppressive therapy in the subject not undergoing or not at risk of undergoing AR. In some embodiments, the nucleic acid is obtained from a peripheral blood sample or a biopsy sample. In some embodiments, application of the algorithm in step (b) generates a performance threshold. In some embodiments, the performance threshold is 0.40, 0.45, 0.50, 0.55, 0.60, 0.65, 0.7, 0.75, or higher.

In some aspects, the disclosure provides a method of prognosing or diagnosing a liver transplant rejection in a subject, comprising: (a) obtaining a sample comprising nucleic acid from the subject; (b) analyzing the nucleic acid to measure expression level of one or more genes listed in Table 3 using nucleic acid obtained from the subject, wherein the one or more genes comprises NHS actin remodeling regulator (NHS), endothelial PAS domain protein 1 (EPAS1), complement component 4 binding protein alpha (C4BPA), lymphocyte cytosolic protein 2 (LCP2), deleted in lymphocytic leukemia 2 (DLEU2), NHS like 1 (NHSL1), required for meiotic nuclear division 5 homolog A (RMND5A), lymphocyte activating 3 (LAG3), chitinase 3 like 2 (CHI3L2), gelsolin (GSN), ASAP1 intronic transcript 2 (ASAP1-IT2), NLR family apoptosis inhibitory protein (NAIP), geminin DNA replication inhibitor (GMNN), C-type lectin domain family 4 member E (CLEC4E), X inactive specific transcript (XIST), long intergenic non-protein coding RNA 877 (LINC00877), A-kinase anchoring protein 12 (AKAP12), ATPase family AAA domain containing 2 (ATAD2), or a combination of any of the foregoing; (c) prognosing or diagnosing the liver transplant rejection in the subject from the expression levels measured in step (b); and (d) treating the subject prognosed or diagnosed with liver transplant rejection with immunosuppressive therapy. In some embodiments, step (a) further comprises determining an expression level of stathmin 1 (STMN1), protein phosphatase 1 regulatory subunit 12B (PPP1R12B), mesoderm specific transcript (MEST), ribonucleotide reductase regulatory subunit M2 (RRM2), senataxin (SETX), thymidylate synthetase (TYMS), GATA binding protein 2 (GATA2), KIAA1324, or a combination of any of the foregoing. In some embodiments, step (a) further comprises determining the expression level of 5, 10, 15, 20, 25, 30, 35, or each of the genes listed in Table 3. In some aspects, the disclosure provides a method of prognosing or diagnosing a liver transplant rejection in a subject, comprising: (a) obtaining a sample comprising nucleic acid from the subject; (b) analyzing the nucleic acid to measure expression level of one or more genes listed in Table 3 using nucleic acid obtained from the subject, wherein the one or more genes is gene number 3, 6, 7, 8, 10, 11, 13, 14, 15, 16, 18, 19, 20, 22, 23, 24, 25, 26, 27, 28, 29, 30, 32, 34, 35, or 36; (c) prognosing or diagnosing the liver transplant rejection in the subject from the expression levels measured in step (b); and (d) treating the subject prognosed or diagnosed with liver transplant rejection with immunosuppressive therapy. In some embodiments, step (a) further comprises determining an expression level of one or more additional genes listed in Table 3, wherein the one or more additional genes is gene number 1, 2, 4, 5, 9, 12, 17, 21, 31, or 33. In some aspects, the disclosure provides a method of prognosing or diagnosing a liver transplant rejection in a subject, comprising: (a) obtaining a sample comprising nucleic acid from the subject; (b) analyzing the nucleic acid to measure expression level of each of the genes listed in Table 3; (c) prognosing or diagnosing the liver transplant rejection in the subject from the expression levels measured in step (b); and (d) treating the subject prognosed or diagnosed with liver transplant rejection with immunosuppressive therapy. In some embodiments, the nucleic acid is mRNA. In further embodiments, the mRNA is used to generate complementary DNA (cDNA) prior to step (b). In some embodiments, a method of the disclosure further comprises contacting the nucleic acid or cDNA with probes, wherein the probes are specific for the one or more genes listed in Table 3. In some embodiments, the sample is a blood sample. In some embodiments, the subject has acute rejection (AR), acute dysfunction no rejection (ADNR), or well-functioning normal transplant (TX). In some embodiments, for each of the genes listed in Table 3, step (c) comprises comparing the expression level of the genes in the subject to one or more reference expression levels of the gene associated with AR, ADNR, or TX. In some embodiments, step (c) further comprises for each of the genes listed in Table 3 assigning the expression level a value or other designation providing an indication whether the subject has AR, ADNR, or TX. In some embodiments, the expression level of each of the genes listed in Table 3 is assigned a value on a normalized scale of values associated with a range of expression levels in liver transplant patients with AR, ADNR, or TX. In some embodiments, the expression level of each of the genes listed in Table 3 is assigned a value or other designation providing an indication that the subject has or is at risk of AR, ADNR, or has well-functioning normal transplant. In some embodiments, step (c) further comprises combining the values or designations for each of the genes to provide a combined value or designation providing an indication whether the subject has or is at risk of AR, ADNR, or has TX. In some embodiments, multiple samples are obtained from the subject over time. In further embodiments, the multiple samples are obtained from the subject at one or more of week 1, week 2, month 1, month 2, month 3, month 6, month 9, month 12, month 15, month 18, month 21, and month 24 after a first sample is obtained. In some embodiments, the subject is receiving a drug, and a change in the combined value or designation over time provides an indication of the effectiveness of the drug. In some embodiments, the subject has undergone a liver transplant within 1 month, 3 months, 1 year, 2 years, 3 years or 5 years of performing step (a). In some embodiments, step (d) represents a change in treatment and is based on the prognosing or diagnosing. In some embodiments, the subject has received a drug before performing the methods, and the change in treatment comprises administering a higher dose of the drug, administering a lower dose of the drug, stopping administration of the drug, administering an alternative drug, or administering an additional drug. In some embodiments, step (c) is performed by a computer. In some embodiments, the expression levels are measured by quantitative PCR, hybridization to an array, or RNA sequencing. In some embodiments, the subject has normal liver function at the time the subject is prognosed or diagnosed with AR or ADNR. In further embodiments, the normal liver function is determined by a liver function test (LFT) in which total bilirubin (TB) is less than 1.5 mg/dL, direct bilirubin is less than 0.5 mg/dL, alkaline phosphatase (AP) is less than 200 U/L, and alanine transaminase (ALT) is less than 60 U/L (males) or less than 36 U/L (females).

In some aspects, the disclosure provides a method of prognosing or diagnosing a liver transplant rejection in a subject, comprising: (a) obtaining a sample comprising nucleic acid from the subject; (b) analyzing the nucleic acid to measure expression level of one or more genes listed in Table 3; (c) prognosing or diagnosing the liver transplant rejection in the subject from the expression levels measured in step (b); and (d) treating the subject prognosed or diagnosed with liver transplant rejection with immunosuppressive therapy. In some embodiments, the nucleic acid is mRNA. In some embodiments, the mRNA is used to generate complementary DNA (cDNA) prior to step (b). In some embodiments, methods of the disclosure further comprise contacting the nucleic acid or cDNA with probes, wherein the probes are specific for the one or more genes listed in Table 3.

In some embodiments, the sample is a blood sample. In some embodiments, the sample is a blood sample or is derived from a blood sample. In further embodiments, the blood sample is a peripheral blood sample. In some embodiments, the blood sample is a whole blood sample. In some embodiments, the sample does not comprise tissue from a biopsy of a transplanted organ of the transplant recipient. In further embodiments, the sample is not derived from tissue from a biopsy of a transplanted organ of the transplant recipient. In further embodiments, the subject has acute rejection (AR), acute dysfunction no rejection (ADNR), or well-functioning normal transplant (TX). In still further embodiments, for each of the genes listed in Table 3, step (c) comprises comparing the expression level of the genes in the subject to one or more reference expression levels of the gene associated with AR, ADNR, or TX. In some embodiments, step (c) further comprises for each of the genes listed in Table 3 assigning the expression level a value or other designation providing an indication whether the subject has AR, ADNR, or TX. In some embodiments, the expression level of each of the genes listed in Table 3 is assigned a value on a normalized scale of values associated with a range of expression levels in liver transplant patients with AR, ADNR, or TX. In further embodiments, the expression level of each of the genes listed in Table 3 is assigned a value or other designation providing an indication that the subject has or is at risk of AR, ADNR, or has well-functioning normal transplant (TX). In some embodiments, step (c) further comprises combining the values or designations for each of the genes to provide a combined value or designation providing an indication whether the subject has or is at risk of AR, ADNR, or has TX. In some embodiments, multiple samples are obtained from the subject over time. In further embodiments, the multiple samples are obtained from the subject at one or more of week 1, week 2, month 1, month 2, month 3, month 6, month 9, month 12, month 15, month 18, month 21, and month 24 after a first sample is obtained. In some embodiments, the subject is receiving a drug, and a change in the combined value or designation over time provides an indication of the effectiveness of the drug. In further embodiments, the subject has undergone a liver transplant within 1 month, 3 months, 1 year, 2 years, 3 years or 5 years of performing step (a). In still further embodiments, step (d) represents a change in treatment and is based on the prognosing or diagnosing. In some embodiments, the subject has received a drug before performing the methods, and the change in treatment comprises administering a higher dose of the drug, administering a lower dose of the drug, stopping administration of the drug, administering an alternative drug, or administering an additional drug. In some embodiments, step (c) is performed by a computer. In some embodiments, the expression levels are measured by quantitative PCR, hybridization to an array, or RNA sequencing.

In some aspects, the disclosure provides a method of screening a compound for activity in inhibiting or treating a liver transplant rejection or injury, comprising: (a) administering the compound to a subject having or at risk of developing the liver transplant rejection or injury; (b) determining expression level of one or more genes listed in Table 3 and species variants thereof before and after administering the compound to the subject; and (c) determining whether the compound has activity in inhibiting or treating the liver transplant rejection or injury from a change in expression levels of the one or more genes after administering the compound. In some embodiments, step (c) comprises for each change in expression level assigning a value or designation depending on whether the change in expression level of the gene relative to one or more reference levels indicating presence or absence of the liver transplant rejection or injury. In some embodiments, the methods further comprise determining a combined value or designation for the one or more genes from the values or designations determined for each gene. In some embodiments, the subject is human or a nonhuman animal model of the liver transplant rejection or injury.

In some aspects, the disclosure provides an array, comprising a support or supports bearing a plurality of nucleic acid probes complementary to a plurality of mRNAs fewer than 5000 in number, wherein the plurality of mRNAs includes mRNAs expressed by one or more genes listed in Table 3. In some embodiments, the plurality of mRNAs are fewer than 1000, fewer than 100, fewer than 50, or fewer in 30 in number. In further embodiments, the plurality of nucleic acid probes are attached to a planar support or to beads.

In some aspects, the disclosure provides an array, comprising a support or supports bearing a plurality of ligands that specifically bind to a plurality of proteins fewer than 5000 in number, wherein the plurality of proteins includes one or more proteins encoded by genes listed in Table 3. In some embodiments, the plurality of proteins are fewer than 1000, fewer than 100, fewer than 50, or fewer in 30 in number. In further embodiments, the plurality of ligands are attached to a planar support or to beads. In still further embodiments, the ligands are different antibodies, wherein the different antibodies bind to different proteins of the plurality of proteins.

In some aspects, the disclosure provides a method of expression analysis, comprising determining expression levels of up to 5000 genes in a sample from a subject having a liver transplant, wherein the genes include one or more of the genes selected from at least one of Table 3. In some embodiments, the expression levels of up to 10, 100 or 1000 genes are determined. In further embodiments, the expression levels are determined at the mRNA level or at the protein level. In still further embodiments, the expression levels are determined by quantitative PCR or hybridization to an array. In some embodiments, the expression levels are determined by a sequencing assay. In some embodiments, the assay is a microarray, SAGE, blotting, RT-PCR, sequencing and/or quantitative PCR assay. In some embodiments, the assay is a microarray assay. In further embodiments, the microarray assay comprises the use of an Affymetrix Human Genome U133 Plus 2.0GeneChip. In some embodiments, the microarray uses the Hu133 Plus 2.0 cartridge arrays plates. In further embodiments, the microarray uses the HT HG-U133+PM array plates. In some embodiments, the assay is a sequencing assay. In further embodiments, the assay is a RNA sequencing assay. A further understanding of the nature and advantages of the present disclosure may be realized by reference to the remaining portions of the specification and claims.

### BRIEF DESCRIPTION OF THE FIGURES

FIG. 1 depicts the CTOT-14 consort flow diagram—Enrollment and Clinical Phenotypes with Samples.

FIG. 2 shows the Receiver Operating Curves (ROC) with Area under the Curve (AUC) for the Blood—AR vs. TX (NU discovery cohort).

FIG. 3 shows serial changes in gene expression using line slopes prior to AR, TX and non-AR. FIG. 3a: pre-AR vs. pre-TX (p<0.001). FIG. 3b: pre-AR vs. pre-non-AR (p<0.001). FIG. 3c: pre-AR vs. post-AR, p=0.081). Of note, the p-value result is the phenotype comparison of the entire line slope from the time of transplantation, whereas the figures visually display a more focused time period around AR, TX, and non-AR diagnosis (Time 0).

### DETAILED DESCRIPTION

It is not possible to predict which patients will develop acute rejection (AR) after liver transplantation (LT). Yet the implications of a LT recipient developing AR are clinically significant as this problem has been associated with an increased risk of death and graft failure. In independent discovery and validation cohorts, the present disclosure exemplifies validation of a blood-based biomarker model containing 36 gene probes that can predict AR before it occurs and is different than patients with stable graft function (TX=Transplant eXcellent) and other causes of dysfunction (ADNR; acute dysfunction/no rejection). This test involves a simple blood collection that could be used to monitor patients for rejection in clinical practice to guide immunosuppressive therapy management.

Applications of the technologies disclosed herein include, but are not limited to, identification of patients likely to develop acute rejection before it occurs. This information is used for immunosuppressive therapy decision-making, such as not lowering the doses if acute rejection is predicted, and therefore reducing rejection overall. Further, the technologies provide the ability to identify patients likely to not develop acute rejection (TX or non-AR) such that immunosuppressive therapy can be more safely lowered. This is important because immunosuppressive therapy has toxicities which can be reduced by lowering doses, as long as rejection does not occur. The test is further useful, in additional embodiments, to allow for continued immunosuppressive therapy lowering as long as TX is predicted and not AR, and therefore reducing the overall toxicity of therapy.

The present disclosure provides advantages to the field because the only predictions available now for estimating the risk of acute rejection are subjective clinical variables and assessments, such as age and cause of liver disease. These can be inaccurate leading to guessing wrong, such as blindly lowering or increasing therapy and potentially leading to rejection or immunosuppression toxicity, respectively. A goal of the blood-based test disclosed herein is to be able to more objectively guide individual LT recipient's immunosuppression requirements. The integration of the biomarkers disclosed herein into clinical practice help diagnose and predict 1) acute rejection 2) healthy transplant (TX), as well as monitor after acute rejection for AR gene expression resolution.

### Definitions

Transplantation is the transfer of tissues, cells or an organ from a donor into a recipient. If the donor and recipient as the same person, the graft is referred to as an autograft and as is usually the case between different individuals of the same species an allograft. Transfer of tissue between species is referred to as a xenograft.

In some embodiments, methods of the disclosure are performed in a subject that has already been diagnosed with, e.g., acute rejection (AR) via conventional criteria. Examples of conventional criteria for diagnosing the various clinical phenotypes are as follows. The clinical phenotype of acute rejection (AR) in a subject is characterized by a ‘for cause biopsy’ due to abnormal liver function tests (LFTs) (acute change) and a central biopsy read that is consistent with AR. ‘Normal’ LFTs are, e.g., tests in which Total Bilirubin (TB)<1.5 mg/dL and Direct Bilirubin <0.5 mg/dL, Alkaline Phosphatase (AP)<200 U/L, and Alanine Transaminase (ALT) <60 U/L (males)<36 U/L (females). The clinical phenotype of acute dysfunction no rejection (ADNR) in a subject is characterized by a ‘for cause biopsy’ for abnormal LFTs (acute change) and a central biopsy read that is inconsistent with etiology other than AR. ‘Normal’ LFTs are as indicated above. The clinical phenotype of well-functioning normal transplant (TX) is defined as normal LFTs at the time of ‘virtual biopsy’. For example, LFTs are obtained 3 months before and 3 months after the ‘virtual biopsy’. ‘Normal’ LFTs are as indicated above. Subjects identified with a clinical phenotype of “ADNR” or “TX” may also collectively be referred to as “non-AR” subjects. The clinical phenotype of pre-AR is defined as a subject having more than 1 sample(s) collected prior to for cause liver biopsy demonstrating AR (as defined above); and at least 2 of 3 liver tests (TB, AP, ALT) were normal at the time of the sample collection. The clinical phenotype of pre-ADNR is defined by the same criteria as pre-AR except the subject has a for-cause biopsy demonstrating ADNR as described above. The clinical phenotype of pre-TX is defined by the same criteria as pre-AR except the subject has a biopsy that meets the TX criteria as described above. Subjects identified with a clinical phenotype of “pre-ADNR” or “pre-TX” may also collectively be referred to as “pre-non-AR” subjects. A “post-AR” subject is one in which resolution of AR is determined, as defined by normal LFTs as described above.

A biopsy is a specimen obtained from a living patient for diagnostic evaluation. Liver biopsies can be obtained with a needle.

A gene expression level is associated with a particular phenotype e.g., presence of a specific liver transplant rejection, if the gene is differentially expressed in a patient having the phenotype relative to a patient lacking the phenotype to a statistically significant extent. Unless otherwise apparent from the context a gene expression level can be measured at the mRNA and/or protein level.

A target nucleic acid is a nucleic acid (often derived from a biological sample), to which a polynucleotide probe is designed to specifically hybridize. The probe can detect presence, absence and/or amount of the target. The term can refer to the specific subsequence of a larger nucleic acid to which the probe is directed or to the overall sequence (e.g., cDNA or mRNA) whose expression level is to be detected. The term can also refer to a nucleic acid that is analyzed by a method, including sequencing, PCR, or other method known in the art.

The term subject or patient can include human or non-human animals. Thus, the methods and described herein are applicable to both human and veterinary disease and animal models. Preferred subjects are “patients,” i.e., living humans that are receiving medical care for a disease or condition. This includes persons with no defined illness who are being investigated for signs of pathology. The term subject or patient can include transplant recipients or donors or healthy subjects. The subjects may be mammals or non-mammals. In some cases, the methods provided herein are used on a subject who has not yet received a transplant, such as a subject who is awaiting a tissue or organ transplant. In other cases, the subject is a transplant donor. In some cases, the subject has not received a transplant and is not expected to receive such transplant. In some cases, the subject maybe a subject who is suffering from diseases requiring monitoring of certain organs for potential failure or dysfunction. In some cases, the subject may be a healthy subject.

Often, the subject is a patient or other individual undergoing a treatment regimen, or being evaluated for a treatment regimen (e.g., immunosuppressive therapy). However, in some instances, the subject is not undergoing a treatment regimen. A feature of the graft tolerant phenotype detected or identified by the methods disclosed herein is that it is a phenotype which occurs without immunosuppressive therapy, e.g., it is present in a subject that is not receiving immunosuppressive therapy.

In various embodiments, the subjects suitable for methods of the disclosure are patients who have undergone an organ transplant within 6 hours, 12 hours, 1 day, 2 days, 3 days, 4 days, 5 days, 10 days, 15 days, 20 days, 25 days, 1 month, 2 months, 3 months, 4 months, 5 months, 7 months, 9 months, 11 months, 1 year, 2 years, 4 years, 5 years, 10 years, 15 years, 20 years or longer of prior to receiving a classification obtained by the methods disclosed herein, such as detection of liver transplant rejection.

Diagnosis refers to methods of estimating or determining whether or not a patient is suffering from a given disease or condition or severity of the condition. Diagnosis does not require ability to determine the presence or absence of a particular disease with 100% accuracy, or even that a given course or outcome is more likely to occur than not. Instead, the “diagnosis” refers to an increased probability that a certain disease or condition is present in the subject compared to the probability before the diagnostic test was performed. Similarly, a prognosis signals a probability that a given course or outcome will occur in a patient relative to the probability before the prognostic test.

A probe or polynucleotide probe is a nucleic acid capable of binding to a target nucleic acid of complementary sequence through one or more types of chemical bonds, usually through complementary base pairing, usually through hydrogen bond formation, thus forming a duplex structure. A probe can include natural (e.g., A, G, C, U, or T) or modified bases (e.g., 7-deazaguanosine, inosine). A probe can be an oligonucleotide which is a single-stranded DNA. Polynucleotide probes can be synthesized or produced from naturally occurring polynucleotides. In addition, the bases in a probe can be joined by a linkage other than a phosphodiester bond, so long as it does not interfere with hybridization. Thus, probes can include, for example, peptide nucleic acids in which the constituent bases are joined by peptide bonds rather than phosphodiester linkages (see, e.g., Nielsen et al., Science 254, 1497-1500 (1991)). Some probes can have leading and/or trailing sequences of noncomplementarity flanking a region of complementarity.

A perfectly matched probe has a sequence perfectly complementary to a particular target sequence. The probe is typically perfectly complementary to a portion (subsequence) of a target sequence. The term “mismatch probe” refer to probes whose sequence is deliberately selected not to be perfectly complementary to a particular target sequence.

The term “isolated,” “purified” or “substantially pure” means an object species (e.g., a nucleic acid sequence described herein or a polypeptide encoded thereby) has been at least partially separated from the components with which it is naturally associated.

Differential expression refers to a statistically significant difference in expression levels of a gene between two populations of samples (e.g., samples with and without a specific transplant rejection). The expression levels can differ for example by a factor of about 0.5, 1, 1.5, 2, or more between such populations of samples. Differential expression includes genes that are expressed in one population and are not expressed (at least at detectable levels) in the other populations. Unique expression, usually associated with proteomic and next-generation sequencing technologies, refers to detectable expression in one population and undetectable expression (i.e., insignificantly different from background) in the other population using the same technique.

Control populations for comparison with populations undergoing a liver transplant rejection or injury are usually referred to as being without acute rejection and have a well-functioning graft. In some embodiments, such a control population also means subjects without ADNR and/or HCV infection.

Hybridization reactions are preferably performed under stringent conditions in which probes or primers hybridize to their intended target with which they have perfect complementarity and not to or at least to a reduced extent to other targets. An example of stringent hybridization conditions are hybridization in 6× sodium chloride/sodium citrate (SSC) at about 45° C., followed by one or more washes in 0.2×SSC, 0.1% SDS at 50° C., 55° C., 60° C., 65° C., or higher.

### Genes in Profiles

The present disclosure exemplifies differentially expressed genes that distinguish different graft injury or condition in liver transplant patients. Specifically, Table 3 lists 36 differentially expressed genes in blood samples based on a 3-way comparison of acute rejection (AR) vs. acute dysfunction no rejection (ADNR) vs. transplant excellent (TX) (i.e., healthy transplant or well-functioning normal transplant). The columns in the table have the following meanings: column 1 is a number assigned to a gene, column 2 is an Affymetrix number indicating a set of probes suitable for measuring expression of the gene, column 3 is a gene symbol, column 4 is a gene name (recognized names of HUGO or similar bodies are used when available), column 5 is the location of the gene product, and columns 6 and 7 show the direction and magnitude of gene expression. As detailed in the Examples herein, these probe sets and corresponding genes are able to distinguish the phenotypes of the above three different types of liver transplants with very high predictive accuracy. The disclosure provides methods of using these genes to accurately distinguish the three noted phenotypes of liver transplant. In addition to expression profiles obtained from blood samples, the disclosure also identifies differentially expressed genes in liver biopsies from transplant patients with different phenotypes.

TABLE 3
Probesets for distinguishing between AR and TX.

| | | | | | Log Fold Change | Log Fold Change |
|---|---|---|---|---|---|---|
| GENE # | ID | SYMBOL | ENTREZ GENE NAME | LOCATION | (AR/TX) | (AR/ADNR) |
| 1 | 217714_PM_X_AT | STMN1 | STATHMIN 1 | Cytoplasm | 0.598102762 | 0.262734907 |
| 2 | 233700_PM_AT | PPP1R12B | PROTEIN PHOSPHATASE 1 | Cytoplasm | −0.905378045 | −0.25976984 |
| | | | REGULATORY SUBUNIT 12B | | | |
| 3 | 233263_PM_AT | unidentified | N/A | | −0.509725068 | −0.312740615 |
| 4 | 202016_PM_AT | MEST | MESODERM SPECIFIC | Cytoplasm | 0.543696437 | 0.259936021 |
| | | | TRANSCRIPT | | | |
| 5 | 241391_PM_AT | unidentified | N/A | | −0.611242782 | −0.242805807 |
| 6 | 242800_PM_AT | NHS | NHS ACTIN REMODELING | Nucleus | −0.668760971 | −0.055646547 |
| | | | REGULATOR | | | |
| 7 | 200878_PM_AT | EPAS1 | ENDOTHELIAL PAS DOMAIN | Nucleus | 0.588503631 | 0.48338859 |
| | | | PROTEIN 1 | | | |
| 8 | 240765_PM_AT | unidentified | N/A | | −0.447735132 | −0.29436907 |
| 9 | 209773_PM_S_AT | RRM2 | RIBONUCLEOTIDE REDUCTASE | Nucleus | 0.967267674 | 0.516531643 |
| | | | REGULATORY SUBUNIT M2 | | | |
| 10 | 205654_PM_AT | C4BPA | COMPLEMENT COMPONENT 4 | Extracellular | 1.101224469 | 0.897920852 |
| | | | BINDING PROTEIN ALPHA | Space | | |
| 11 | 1560552_PM_A_AT | unidentified | N/A | | −0.819147799 | −0.391574931 |
| 12 | 233957_PM_AT | unidentified | N/A | | −0.725537325 | −0.240100487 |
| 13 | 236409_PM_AT | unidentified | N/A | | −0.44992291 | −0.249331692 |
| 14 | 244578_PM_AT | LCP2 | LYMPHOCYTE CYTOSOLIC | Cytoplasm | −0.651164906 | −0.257749646 |
| | | | PROTEIN 2 | | | |
| 15 | 242854_PM_X_AT | DLEU2 | DELETED IN LYMPHOCYTIC | Other | −0.654282894 | −0.274431034 |
| | | | LEUKEMIA 2 | | | |
| 16 | 231034_PM_S_AT | NHSL1 | NHS LIKE 1 | Other | −0.534151178 | −0.227014882 |
| 17 | 232229_PM_AT | SETX | SENATAXIN | Nucleus | −0.593424521 | −0.172365985 |
| 18 | 212478_PM_AT | RMND5A | REQUIRED FOR MEIOTIC | Nucleus | −0.333262502 | −0.35407151 |
| | | | NUCLEAR DIVISION 5 | | | |
| | | | HOMOLOG A | | | |
| 19 | 206486_PM_AT | LAG3 | LYMPHOCYTE ACTIVATING 3 | Plasma | 0.312979113 | 0.149092455 |
| | | | | Membrane | | |
| 20 | 213060_PM_S_AT | CHI3L2 | CHITINASE 3 LIKE 2 | Extracellular | 0.653342435 | 0.364803907 |
| | | | | Space | | |
| 21 | 1554696_PM_S_AT | TYMS | THYMIDYLATE SYNTHETASE | Nucleus | 0.558038596 | 0.372408001 |
| 22 | 234431_PM_AT | GSN | GELSOLIN | Extracellular | −0.36530257 | −0.112941939 |
| | | | | Space | | |
| 23 | 1557685_PM_AT | ASAP1-IT2 | ASAP1 INTRONIC TRANSCRIPT 2 | Other | −0.563114239 | −0.004750863 |
| 24 | 238446_PM_AT | NAIP | NLR FAMILY APOPTOSIS | Cytoplasm | −0.756993933 | −0.460497962 |
| | | | INHIBITORY PROTEIN | | | |
| 25 | 237376_PM_AT | unidentified | N/A | | −0.525639819 | −0.250703138 |
| 26 | 218350_PM_S_AT | GMNN | GEMININ DNA REPLICATION | Nucleus | 0.600891795 | 0.192253388 |
| | | | INHIBITOR | | | |
| 27 | 219859_PM_AT | CLEC4E | C-TYPE LECTIN DOMAIN FAMILY 4 | Plasma | 0.398082121 | 0.207993257 |
| | | | MEMBER E | Membrane | | |
| 28 | 238281_PM_AT | unidentified | N/A | | −0.323326791 | −0.253762725 |
| 29 | 227671_PM_AT | XIST | X INACTIVE SPECIFIC | Nucleus | 2.556868022 | 1.325410826 |
| | | | TRANSCRIPT | | | |
| 30 | 243954_PM_AT | LINC00877 | LONG INTERGENIC NON-PROTEIN | Other | −0.880361117 | −0.095247824 |
| | | | CODING RNA 877 | | | |
| 31 | 210358_PM_X_AT | GATA2 | GATA BINDING PROTEIN 2 | Nucleus | 0.511546109 | 0.477037794 |
| 32 | 236216_PM_AT | unidentified | N/A | | −0.635686036 | 0.013480067 |
| 33 | 221874_PM_AT | KIAA1324 | KIAA1324 | Plasma | −1.001702606 | −0.692655875 |
| | | | | Membrane | | |
| 34 | 243874_PM_AT | unidentified | N/A | | −0.720702961 | −0.167061836 |
| 35 | 227530_PM_AT | AKAP12 | A-KINASE ANCHORING PROTEIN | Cytoplasm | 0.511682439 | 0.405332578 |
| | | | 12 | | | |
| 36 | 218782_PM_S_AT | ATAD2 | ATPASE FAMILY AAA DOMAIN | Nucleus | 0.389173008 | 0.191528324 |
| | | | CONTAINING 2 | | | |

The genes referred to in the above table are human genes. In some methods, species variants or homologs of these genes are used in anon-human animal model. Species variants are the genes in different species having greatest sequence identity and similarity in functional properties to one another. Many species variants of the above human genes are listed in the Swiss-Prot database.

To identify differentially expressed genes, raw gene expression levels are comparable between different genes in the same sample but not necessarily between different samples. Values given for gene expression levels can be normalized so that values for particular genes are comparable within and between the populations being analyzed. The normalization eliminates or at least reduces to acceptable levels any sample to sample differences arising from factors other than a specific type of liver transplant rejection or injury (e.g. differences in overall transcription levels of patients due to general state of health and differences in sample preparation or nucleic acid amplification between samples). The normalization effectively applies a correction factor to the measured expression levels from a given array such that a profile of many expression levels in the array are the same between different patient samples. Software for normalizing overall expression patterns between different samples is both commercially and publically available (e.g., Partek Genomics Suite from Partek, XRAY from Biotique Systems or BRB ArrayTools from the National Cancer Institute). After applying appropriate normalizing factors to the measured expression value of a particular gene in different samples, an average or mean value of the expression level is determined for the samples in a population. The average or mean values between different populations are then compared to determine whether expression level has changed significantly between the populations. The changes in expression level indicated for a given gene represent the relative expression level of that gene in samples from a population of individuals with a defined condition (e.g., transplant patients with acute rejection) relative to samples from a control population (liver transplant patients not undergoing rejection). Similar principles apply in normalizing gene expression levels at the mRNA and protein levels. Comparisons between populations are made at the same level (e.g., mRNA levels in one population are compared with mRNA levels in another population or protein levels in one population with protein levels in another population).

### Study Populations

The methods described herein are particularly useful on human subjects who have undergone a liver transplant although can also be used on subjects who have undergone other types of transplant (e.g., heart, kidney, lungs, stem cell) or on non-humans who have undergone liver or other transplant. The patients may have or are at risk of developing any of the phenotypes of graft rejection or injuries described herein. These include patients with acute rejection (AR), patients with acute dysfunction no rejection (ADNR), and patients who have normal functional graft or transplant excellent (TX).

Patients with phenotypes of graft rejection or injuries described herein can be diagnosed through biopsies that are taken at a fixed time after transplantation (e.g., protocol biopsies or serial monitoring biopsies) which are not driven by clinical indications but rather by standards of care. The biopsies may be analyzed histologically in order to detect the liver transplant rejection. A failure to recognize, diagnose and treat any of the phenotypes of graft rejection or injuries before significant tissue injury has occurred and the transplant shows clinical signs of dysfunction could be a major cause of irreversible organ damage. Moreover, a failure to recognize chronic, subclinical immune-mediated organ damage and a failure to make appropriate changes in immunosuppressive therapy to restore a state of effective immunosuppression in that patient could contribute to late organ transplant failure. The methods disclosed herein can reduce or eliminate these and other problems associated with transplant rejection or failure. In some embodiments, the subject population comprises liver transplant patients who have acute rejection (AR). In further embodiments, the subject population comprises liver transplant patients who have or are at risk of having acute rejection (AR), have or are at risk of having acute dysfunction no rejection (ADNR), or are transplant excellent (TX).

Acute rejection (AR) or clinical acute rejection may occur when transplanted tissue is rejected by the recipient's immune system, which damages or destroys the transplanted tissue unless immunosuppression is achieved. T-cells, B-cells and other immune cells as well as possibly antibodies of the recipient may cause the graft cells to lyse or produce cytokines that recruit other inflammatory cells, eventually causing necrosis of allograft tissue. In some instances, AR may be diagnosed by a biopsy of the transplanted organ. The treatment of AR may include using immunosuppressive agents, corticosteroids, polyclonal and monoclonal antibodies, engineered and naturally occurring biological molecules, and antiproliferatives. AR more frequently occurs in the first three to 12 months after transplantation but there is a continued risk and incidence of AR for the first five years post-transplant and whenever a patient's immunosuppression becomes inadequate for any reason for the life of the transplant.

The methods of the disclosure may also be used to distinguish between a liver transplant patient with AR and a normally functioning liver transplant. Typically, when the patient does not exhibit symptoms or test results of organ dysfunction or rejection, the transplant is considered a normal functioning transplant (TX: Transplant eXcellent). An unhealthy transplant recipient may exhibit signs of organ dysfunction and/or rejection.

Regardless of the specific subject population, gene expression levels in such subjects can be measured, for example, within, one month, three months, six months, one year, two years, five years or ten years after a liver transplant. In some embodiments, gene expression levels are determined at regular intervals, e.g., every 3 months, 6 months or every year post-transplant, either indefinitely, or until evidence of graft rejection or injury is observed, in which case the frequency of monitoring is sometimes increased. In some embodiments, baseline values of expression levels are determined in a subject before a liver transplant in combination with determining expression levels at one or more time points thereafter. In some embodiments, a measurement is initiated responsive to some other indication of potential liver impairment, such as a rise in levels of creatinine or Blood Urea Nitrogen (BUN) or a decrease in glomerular filtration rate. Similar methods can be practiced in non-human species, in which cases, the expression levels measured are the species equivalent of the human genes referenced above.

### Methods of Measuring Profiles

Samples. Methods of the disclosure can utilize either a blood sample or a biopsy sample from the patient. In some embodiments, a blood sample is used, which can be peripheral whole blood or fractions thereof, such as plasma, or lymphocytes. In some embodiments, a liver biopsy is obtained from the patient for expression profile analysis. Other samples that may be employed in measuring gene expression profiles include but are not limited to urine, feces, and saliva. The samples are typically isolated from a subject and not returned to the subject. The analytes of interests in the samples can be analyzed with or without further processing of the sample, such as purification and amplification. For prognosis or diagnosis of AR inpatients as opposed to patients with ANDR or patients without rejection (TX), the profiles can comprise genes listed in Table 3 or combinations thereof. In these embodiments, a blood sample is preferably used. However, a sample may be any material containing tissues, cells, nucleic acids, genes, gene fragments, expression products, polypeptides, exosomes, gene expression products, or gene expression product fragments of a subject to be tested. In some cases, the sample is from a single patient. In some cases, the method comprises analyzing multiple samples at once, e.g., via massively parallel sequencing. In some embodiments, the profile comprises NHS actin remodeling regulator (NHS), endothelial PAS domain protein 1 (EPAS1), complement component 4 binding protein alpha (C4BPA), lymphocyte cytosolic protein 2 (LCP2), deleted in lymphocytic leukemia 2 (DLEU2), NHS like 1 (NHSL1), required for meiotic nuclear division 5 homolog A (RMND5A), lymphocyte activating 3 (LAG3), chitinase 3 like 2 (CHI3L2), gelsolin (GSN), ASAP1 intronic transcript 2 (ASAP1-IT2), NLR family apoptosis inhibitory protein (NAIP), geminin DNA replication inhibitor (GMNN), C-type lectin domain family 4 member E (CLEC4E), X inactive specific transcript (XIST), long intergenic non-protein coding RNA 877 (LINC00877), A-kinase anchoring protein 12 (AKAP12), ATPase family AAA domain containing 2 (ATAD2), or a combination of any of the foregoing. In further embodiments, the profile further comprises stathmin 1 (STMN1), protein phosphatase 1 regulatory subunit 12B (PPP1R12B), mesoderm specific transcript (MEST), ribonucleotide reductase regulatory subunit M2 (RRM2), senataxin (SETX), thymidylate synthetase (TYMS), GATA binding protein 2 (GATA2), KIAA1324, or a combination of any of the foregoing. In some embodiments, the profile comprises one or more of gene number 3, 6, 7, 8, 10, 11, 13, 14, 15, 16, 18, 19, 20, 22, 23, 24, 25, 26, 27, 28, 29, 30, 32, 34, 35, or 36, each as listed in Table 3. In some embodiments, the profile further comprises one or more of gene number 1, 2, 4, 5, 9, 12, 17, 21, 31, or 33, each as listed in Table 3.

The sample can be blood. In some embodiments, the sample comprises whole blood, plasma, peripheral blood lymphocytes (PBLs), peripheral blood mononuclear cells (PBMCs), serum, T cells, B Cells, CD3 cells, CD8 cells, CD4 cells, and/or other immune cells.

The methods, kits, and systems disclosed herein may comprise specifically detecting, profiling, or quantitating molecules (e.g., nucleic acids, DNA, RNA, polypeptides, etc.) that are within the biological samples. In some instances, genomic expression products, including RNA, or polypeptides, may be isolated from the biological samples. In some cases, nucleic acids, DNA, RNA, polypeptides may be isolated from a cell-free source. In some cases, nucleic acids, DNA, RNA, polypeptides may be isolated from cells derived from the transplant recipient.

The sample may be obtained using any method known to the art that can provide a sample suitable for the analytical methods described herein. The sample may be obtained by a non-invasive method such as a throat swab, buccal swab, bronchial lavage, urine collection, scraping of the skin or cervix, swabbing of the cheek, saliva collection, feces collection, menses collection, or semen collection.

The sample may be obtained by a minimally-invasive method such as a blood draw. The sample may be obtained by venipuncture. In other instances, the sample is obtained by an invasive procedure including but not limited to: biopsy, alveolar or pulmonary lavage, or needle aspiration. The method of biopsy may include surgical biopsy, incisional biopsy, excisional biopsy, punch biopsy, shave biopsy, or skin biopsy. The sample may be formalin fixed sections. The method of needle aspiration may further include fine needle aspiration, core needle biopsy, vacuum assisted biopsy, or large core biopsy. In some embodiments, multiple samples may be obtained by the methods herein to ensure a sufficient amount of biological material. In some instances, the sample is not obtained by biopsy. In some instances, the sample is not a liver biopsy.

Expression Profiles. Methods provided by the disclosure include those that are directed to prognosis or diagnosis to distinguish patients who have or are at risk of developing AR, and patients without rejection (TX). For these methods, the genes in the expression profiles to be measured can be selected from Table 3. In some embodiments, a blood sample is preferably used. Such methods preferably utilize an expression profile of genes selected from Table 3. In some embodiments, a liver biopsy sample is preferably used.

Expression profiles are preferably measured at the nucleic acid level, meaning that levels of mRNA or nucleic acid derived therefrom (e.g., cDNA or cRNA). An expression profile refers to the expression levels of a plurality of genes in a sample. A nucleic acid derived from mRNA means a nucleic acid synthesized using mRNA as a template. Methods of isolation and amplification of mRNA are well known in the art, e.g., as described in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993). If mRNA or a nucleic acid therefrom is amplified, the amplification is performed under conditions that approximately preserve the relative proportions of mRNA in the original samples, such that the levels of the amplified nucleic acids can be used to establish phenotypic associations representative of the mRNAs.

A variety of approaches are available for determining mRNA levels including probe arrays and quantitative PCR. A number of distinct array formats are available. Some arrays, such as an Affymetrix HG-U133 PM microarray or other Affymetrix GeneChip® array, have different probes occupying discrete known areas of a contiguous support. Exemplary microarrays include but are not limited to the Affymetrix Human GenomeU133 Plus 2.0 GeneChip or the HT HG-U133+PM Array Plate.

Other arrays, such as arrays from Illumine, have different probes attached to different particles or beads. In such arrays, the identity of which probe is attached to which particle or beads is usually determinable from an encoding system. The probes can be oligonucleotides. In such case, typically several match probes are included with perfect complementarity to a given target mRNA together, optionally together with mismatch probes differing from the match probes are a known number of oligonucleotides (Lockhart, et al., Nature Biotechnology 14:1675-1680(1996); and Lipschutz, et al., Nature Genetics Supplement 21: 20-24, 1999). Other arrays including full length cDNA sequences with perfect or near perfect complementarity to a particular cDNA (Schena et al. (Science 270:467-470 (1995); and DeRisi et al. (Nature Genetics 14:457-460(1996)). Such arrays can also include various control probes, such as a probe complementary to a house keeping gene likely to be expressed in most samples. Regardless of the specifics of array design, an array contains one or more probes either perfectly complementary to a particular target mRNA or sufficiently complementary to the target mRNA to distinguish it from other mRNAs in the sample, and the presence of such a target mRNA can be determined from the hybridization signal of such probes, optionally by comparison with mismatch or other control probes included in the array. Typically, the target bears a fluorescent label, in which case hybridization intensity can be determined by, for example, a scanning confocal microscope in photon counting mode. Appropriate scanning devices are described by e.g., U.S. Pat. Nos. 5,578,832, and 5,631,734. The intensity of labeling of probes hybridizing to a particular mRNA or its amplification product provides a raw measure of expression level.

In some embodiments, expression levels are determined by so-called “real time amplification” methods also known as quantitative PCR or Taqman (see, e.g., U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994, 1996; Gibson, U. E. M, et al., Genome Research 6:995-1001, 1996; Holland, P. M., et al., Proc. Natl. Acad. Sci. USA 88:7276-7280, 1991; and Livak, K. J., et al., PCR Methods and Applications 357-362, 1995). The basis for this method of monitoring the formation of amplification product is to continuously measure PCR product accumulation using a dual-labeled fluorogenic oligonucleotide probe. The probe used in such assays is typically a short (approximately 20-25 bases) polynucleotide that is labeled with two different fluorescent dyes. The 5′ terminus of the probe is typically attached to a reporter dye and the 3′ terminus is attached to a quenching dye. The probe is designed to have at least substantial sequence complementarity with a site on the target mRNA or nucleic acid derived from. Upstream and downstream PCR primers that bind to flanking regions of the locus are also added to the reaction mixture. When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector. The recorded values can then be used to calculate the increase in normalized reporter emission intensity on a continuous basis and ultimately quantify the amount of the mRNA being amplified. mRNA levels can also be measured without amplification by hybridization to a probe, for example, using a branched nucleic acid probe, such as a QuantiGene® Reagent System from Panomics.

In some embodiments, the expression level of the gene products (e.g., RNA) is determined by sequencing, such as by RNA sequencing or by DNA sequencing (e.g., of cDNA generated from reverse-transcribing RNA (e.g., mRNA) from a sample). Sequencing may be performed by any available method or technique. Sequencing methods may include: Next Generation sequencing, high-throughput sequencing, pyrosequencing, classic Sanger sequencing methods, sequencing-by-ligation, sequencing by synthesis, sequencing-by-hybridization, RNA-Seq (Illumine), Digital Gene Expression (Helicos), next generation sequencing, single molecule sequencing by synthesis (SMSS) (Helicos), Ion Torrent Sequencing Machine (Life Technologies/Thermo-Fisher), massively-parallel sequencing, clonal single molecule Array (Solexa), shotgun sequencing, Maxim-Gilbert sequencing, primer walking, and any other sequencing methods known in the art.

Measuring gene expression levels may comprise reverse transcribing RNA (e.g., mRNA) within a sample in order to produce cDNA. The cDNA may then be measured using any of the methods described herein (e.g., PCR, digital PCR, qPCR, microarray, SAGE, blotting, sequencing, etc.).

Alternatively or additionally, expression levels of genes can be determined at the protein level, meaning that levels of proteins encoded by the genes discussed above are measured. Several methods and devices are well known for determining levels of proteins including immunoassays such as described in e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792. These assays include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays typically include different antibodies having specificity for different proteins intended to be detected. For example, usually at least one hundred different antibodies are used to detect one hundred different protein targets, each antibody being specific for one target. Other ligands having specificity for a particular protein target can also be used, such as the synthetic antibodies disclosed in WO/2008/048970. Other compounds with a desired binding specificity can be selected from random libraries of peptides or small molecules. U.S. Pat. No. 5,922,615 describes a device that utilizes multiple discrete zones of immobilized antibodies on membranes to detect multiple target antigens in an array. U.S. Pat. Nos. 5,458,852, 6,019,944, U.S. Pat. No. 6,143,576. Microtiter plates or automation can be used to facilitate detection of large numbers of different proteins. Protein levels can also be determined by mass spectrometry.

The selection of genes for determination of expression levels depends on the particular application. In general, the genes are selected from a table of the disclosure as appropriate for the application. In some methods, expression levels of at least 2, 3, 4, 5, 10, 15, 20, 25, 30, or 35 (e.g., 10-30) genes shown in Table 3 are determined. In some embodiments, expression levels of at least 2, 3, 4, 5, 6, 7. 8, 9, 10, 11, 12, 13, 14, 15, 16, 17, 18, 19, 20, 21, 22, 23, 24, 25, 26, 27, 28, 29, 30, 31, 32, 33, 34, 35, or all genes shown in Table 3 are determined. In some embodiments, the expression level of about 5, 10, 15, 20, 25, 30, 35, or each of the genes listed in Table 3 is determined. In further embodiments, the expression level of less than 36, 35, 30, 25, 20, 15, 10, or 5 genes listed in Table 3 is determined. In some embodiments, genes are selected such that genes from several different pathways are represented. The genes within a pathway tend to be expressed in a coordinated expression whereas genes from different pathways tend to be expressed more independently. Thus, changes in expression based on the aggregate changes of genes from different pathways can have greater statistical significance than aggregate changes of genes within a pathway. In some embodiments, expression levels of the top 5, top 10, top 15, top 20, top 25, top 30, or top 35, genes listed in Table 3 are determined. As noted above, expression levels can be measured at either mRNA levels or protein levels.

Expression levels of the genes and/or proteins disclosed herein can be combined with or without determination of expression levels of any other genes or proteins of interest (e.g., genes or proteins associated with rejection of livers or other organs, e.g., as described in U.S. Patent Publication No. 2017/0183735, Hama et al., Liver Transpl. 2009 15(5):509-21; Rattanasiri et al., Transpl Immunol. 2013 28(1):62-70; and Spivey et al., J. Translational Med. 2011 9:174, each of which is incorporated herein by reference in its entirety. In some embodiments, the genes in the expression profiles to be measured do not include at least one or all of the genes discussed in Gehrau et al., Mol. Med. 2011; 17(7-8):824-33; Asaoka et al., Liver Transpl. 2009 December; 15(12):1738-49; and Sreekumar et al., Liver Transpl. 2002 September; 8(9):814-21. These include, e.g., genes encoding arginase type II (ARG2), ethylmalonic encephalopathy 1 (ETHE1), transmembrane protein 176A (TMEM176A), TMEM176B, caspase 8, apoptosis-related cysteine peptidase, and bone morphogenetic protein 2, transcription factor ISGF-3, interferon-responsive transcription factor (transcription factors), heat shock protein 70 (stress response/chaperone), ubiquitin-conjugating enzyme E2, ubiquitin, ubiquitin-activating enzyme E1 and granzyme B (protein degradation), nicotinamide N-methyltransferase (nicotinamide metabolism), major histocompatibility complex (MHC) class I and II (immune function), transforming growth factor (TGF)-beta and insulin-like growth factor I (growth factors), glycogen synthase and phosphoenolpyruvate carboxykinase (glucose metabolism), cytidinetriphosphate (CTP) synthetase, medium-chain acyl-CoA dehydrogenase and triglyceride lipase (fatty acid metabolism), complement components C1q and C3 (complement activation), p-selectin (cell adhesion), tumor necrosis factor (TNF)-related apoptosis inducing ligand (TRAIL), TNF-alpha converting enzyme, TNF-alpha inducible protein A20, TNF-alpha (apoptosis), alanyl-tRNA synthetase, ribosomal protein-L8, elongation TU, protein synthesis factor eIF-4C, elongation factor-2, eukaryotic initiation factor-4AI and elongation factor-1 alpha (protein synthesis), chaperonin 10 and protein disulfide isomerase (protein folding), insulin-like growth factor (IGF)-binding protein (growth factor), GLUT-2 (glucose metabolism), very-long-chain acyl CoA dehydrogenase and fatty acid omega hydroxylase (fatty acid metabolism), and MT-1 and glutathione peroxidase (DNA metabolism).

Regardless of the format adopted, the present methods can (but need not) be practiced by detecting expression levels of a relatively small number of genes or proteins. In some embodiments, the total number of genes whose expression levels are determined is less than 5000, 1000, 500, 200, 100, 50, 25, 10, 5 or 3. In some methods, the total number of genes whose expression level is determined is 100-1500, 100-250, 500-1500 or 750-1250. In some methods, the total number of proteins whose expression levels are determined is less than 5000, 1000,500, 200, 100, 50, 25, 10, 5 or 3. In some methods, the total number of proteins whose expression level is determined is 100-1500, 100-250, 500-1500 or 750-1250. Correspondingly, when an array form is used for detection of expression levels, the array includes probes or probes sets for less than 5000, 1000, 500, 200, 100, 50, 25, 10, 5 or 3 genes. Thus, for example, an Affymetrix GeneChip® expression monitoring array contains a set of about 20-50 oligonucleotide probes (half match and half-mismatch) for monitoring each gene of interest. Such an array design would include less than 5000, 1000, 500, 200, 100, 50, 25, 10, 5 or 3 such probes sets for detecting less than 5000, 1000, 500, 200, 100, 50, 25, 10, 5 or 3 genes. By further example, an alternative array including one cDNA for each gene whose expression level is to be detected would contain less than 5000, 1000, 500, 200, 100, 50, 25, 10, 5 or 3 such cDNAs for analyzing less than 5000, 1000, 500, 200, 100, 50, 25, 10, 5 or 3 genes. By further example, an array containing a different antibody for each protein to be detected would containing less than 5000, 1000, 500,200, 100, 50, 25, 10, 5 or 3 different antibodies for analyzing less than 5000, 1000, 500, 200, 100, 50, 25, 10, 5 or 3 gene products.

### Analysis of Expression Levels

Analysis of expression levels initially provides a measurement of the expression level of each of several individual genes. The expression level can be absolute in terms of a concentration of an expression product, or relative in terms of a relative concentration of an expression product of interest to another expression product in the sample. For example, relative expression levels of genes can be expressed with respect to the expression level of a house-keeping gene in the sample. Relative expression levels can also be determined by simultaneously analyzing differentially labeled samples hybridized to the same array. Expression levels can also be expressed in arbitrary units, for example, related to signal intensity.

The individual expression levels, whether absolute or relative, can be converted into values or other designations providing an indication of presence or risk of a liver transplant rejection or injury by comparison with one or more reference points. For different phenotypes of graft injuries (e.g., AR, ADNR, or TX), different gene sets are typically used in the analysis. For example, acute dysfunction no rejection (ADNR) and acute rejection (AR) can be determined via blood samples with gene sets selected from Table 3.

For liver transplant with each of the phenotypes noted above, the reference points can include a measure of an average or mean expression level of a gene in subjects having had a liver transplant with the specific phenotype. The reference points can also include a scale of values found in liver transplant patients including patients having that phenotype. The reference points can also or alternatively include a reference value in the subject before liver transplant, or a reference value in a population of patients who have not undergone liver transplant. Such reference points can be expressed in terms of absolute or relative concentrations of gene products as for measured values in a sample.

For comparison between a measured expression level and reference level(s), the measured level sometimes needs to be normalized for comparison with the reference level(s) or vice versa. The normalization serves to eliminate or at least minimize changes in expression level unrelated to the specific liver transplant injury or phenotype (e.g., from differences in overall health of the patient or sample preparation) or from purely technical artifacts. Normalization can be performed by determining what factor is needed to equalize a profile of expression levels measured from different genes in a sample with expression levels of these genes in a set of reference samples from which the reference levels were determined. Commercial software is available for performing such normalizations between different sets of expression levels.

Comparison of the measured expression level of a gene with one or more of the above reference points provides a value (i.e., numerical) or other designation (e.g., symbol or word(s)) of presence or susceptibility to a liver transplant injury. In some embodiments, a binary system is used; that is, a measured expression level of a gene is assigned a value or other designation indicating presence or susceptibility to a liver transplant injury or lack thereof without regard to degree. For example, the expression level can be assigned a value of 1 to indicate presence or susceptibility to an injury and −1 to indicate absence or lack of susceptibility to the injury. Such assignment can be based on whether the measured expression level is closer to an average or mean level in liver transplant patients having or not having a specific injury phenotype. In some embodiments, a ternary system is used in which an expression level is assigned a value or other designation indicating presence or susceptibility to a specific injury phenotype or lack thereof or that the expression level is uninformative. Such assignment can be based on whether the expression level is closer to the average or mean level in liver transplant patients undergoing the specific injury, closer to an average or mean level in liver transplant patients lacking the injury or intermediate between such levels. For example, the expression level can be assigned a value of +1, −1 or 0 depending on whether it is closer to the average or mean level in patients undergoing the injury, is closer to the average or mean level in patients not undergoing the injury or is intermediate. In other methods, a particular expression level is assigned a value on a scale, where the upper level is a measure of the highest expression level found in liver transplant patients and the lowest level of the scale is a measure of the lowest expression level found in liver transplant patients at a defined time point at which patients may be susceptible to a graft rejection or injury (e.g., one year post transplant). Preferably, such a scale is normalized scale (e.g., from 0-1) such that the same scale can be used for different genes. Optionally, the value of a measured expression level on such a scale is indicated as being positive or negative depending on whether the upper level of the scale associates with presence or susceptibility to the injury or lack thereof. It does not matter whether a positive or negative sign is used for an injury phenotype or lack thereof as long as the usage is consistent for different genes.

Values or other designation can also be assigned based on a change in expression level of a gene relative to a previous measurement of the expression level of gene in the same patient. Here as elsewhere expression level of a gene can be measured at the protein or nucleic acid level. Such a change can be characterized as being toward, away from or neutral with respect to average or mean expression levels of the gene in liver transplant patients undergoing or not undergoing a grant rejection or injury. For example, a gene whose expression level changes toward an average or mean expression level in liver transplant patients undergoing a graft injury can be assigned a value of 1, and a gene whose expression level changes way from an average or mean expression level in liver transplant patients undergoing the injury and toward an average or mean expression level in liver transplant patients not undergoing the injury can be assigned a value −1. Of course, more sophisticated systems of assigning values are possible based on the magnitude of changes in expression of a gene in a patient.

Having determined values or other designations of expression levels of individual genes providing an indication of presence or susceptibility to a liver graft injury or lack thereof, the values or designations may be combined to provide an aggregate value for all of the genes in the signature being analyzed. If each gene is assigned a score of +1 if its expression level indicates presence or susceptibility to a graft injury and −1 if its expression level indicates absence or lack of susceptibility to the injury and optionally zero if uninformative, the different values can be combined by addition. The same approach can be used if each gene is assigned a value on the same normalized scale and assigned as being positive or negative depending whether the upper point of the scale is associate with presence or susceptibility to a specific liver grant injury or lack thereof. The same method can be performed using the signal intensity. In some cases, the signal intensity for each gene is obtained and used to compute a score. The score may be obtained by adding the upregulated genes to obtain an upregulated value and adding the downregulated genes to obtain a downregulated value and then comparing the downregulated value with the upregulated value (e.g., by calculating a ratio) to determine the score. Other methods of combining values for individual markers of disease into a composite value that can be used as a single marker are described in US20040126767 and WO/2004/059293. In some cases, the score may be used to evaluate severity of a transplant condition, such as by comparing the score with a score normally associated with liver transplant rejection. In some cases, the score maybe used to monitor a subject transplant recipient over time. In such case, scores at a plurality of time points may be compared in order to assess the relative condition of the subject. For example, if the subject's score rises over time, that may indicate that the subject has liver transplant rejection and that his or her condition is worsening over time.

Sample Data. The data pertaining to the sample may be compared to data pertaining to one or more control samples, which may be samples from the same patient at different times. In some embodiments, the one or more control samples comprise one or more samples from healthy subjects, unhealthy subjects, or a combination thereof. The one or more control samples may comprise one or more samples from healthy subjects, subjects suffering from transplant dysfunction with no rejection, subjects suffering from transplant rejection, or a combination thereof. The healthy subjects may be subjects with normal transplant function. The data pertaining to the sample may be sequentially compared to two or more classes of samples. The data pertaining to the sample may be sequentially compared to three or more classes of samples. The classes of samples may comprise control samples classified as being from subjects with normal transplant function, control samples classified as being from subjects suffering from transplant dysfunction with no rejection, control samples classified as being from subjects suffering from transplant rejection, or a combination thereof.

Classifiers. The methods of the disclosure include using a trained classifier or algorithm to analyze sample data, particularly to detect liver transplant rejection. In some instances, the expression levels from sample are used to develop or train an algorithm or classifier provided herein. In some instances, gene expression levels are measured in a sample from a transplant recipient (or a healthy or transplant excellent control) and a classifier or algorithm (e.g., trained algorithm) is applied to the resulting data in order to detect, predict, monitor, or estimate the risk of a transplant condition (e.g., liver transplant rejection). In some embodiments, the algorithm described herein is a trained algorithm. In further embodiments, the trained algorithm is trained with gene expression data from biological samples from at least three different cohorts. In some embodiments, the trained algorithm comprises a linear classifier. In further embodiments, the linear classifier comprises one or more linear discriminant analysis, Fisher's linear discriminant, Naive Bayes classifier, Logistic regression, Perceptron, Support vector machine (SVM) or a combination thereof. In some embodiments, the algorithm comprises a Diagonal Linear Discriminant Analysis (DLDA) algorithm. In some embodiments, the algorithm comprises a Nearest Centroid algorithm. In some embodiments, the algorithm comprises a Random Forest algorithm or statistical bootstrapping. In further embodiments, the algorithm comprises a Prediction Analysis of Microarrays (PAM) algorithm. In some embodiments, the algorithm is not validated by a cohort-based analysis of an entire cohort. In some embodiments, the algorithm is validated by a combined analysis with an unknown phenotype and a subset of a cohort with known phenotypes.

Training of multi-dimensional classifiers (e.g., algorithms) may be performed using numerous samples. For example, training of the multi-dimensional classifier may be performed using at least about 10, 20, 30, 40, 50, 60, 70, 80, 90, 100, 110, 120, 130, 140, 150, 160, 170,180, 190, 200 or more samples. In some embodiments, training of the multi-dimensional classifier may be performed using at least about 200, 210, 220, 230, 240, 250, 260, 270, 280, 290, 300, 350, 400, 450, 500 or more samples. In some embodiments, training of the multi-dimensional classifier may be performed using at least about 525, 550, 600, 650, 700, 750, 800,850, 900, 950, 1000, 1100, 1200, 1300, 1400, 1500, 1600, 1700, 1800, 2000 or more samples.

Further disclosed herein are classifier sets and methods of producing one or more classifier sets. The classifier set may comprise one or more genes, particularly genes from Table 3. In some cases, the classifier set may comprise 1, 2, 3, 4, 5, 6, 7. 8, 9, 10, 11, 12, 13, 14, 15, 16, 17, 18, 19, 20, 21, 22, 23, 24, 25, 26, 27, 28, 29, 30, 31, 32, 33, 34, 35, or more genes from Table 3. Disclosed herein is the use of a classification system comprising one or more classifiers. In some embodiments, the classifier is a 2-, 3-, 4-, 5-, 6-, 7-, 8-, 9-, or 10-way classifier. In some embodiments, the classifier is a 15-, 20-, 25-, 30-, 35-, 40-, 45-, 50-, 55-, 60-, 65-, 70-, 75-, 80-, 85-, 90-, 95-, or 100-way classifier. In some embodiments, the classifier is a three-way classifier. In some embodiments, the classifier is a four-way classifier.

A two-way classifier may classify a sample from a subject into one of two classes. In some embodiments, a two-way classifier may classify a sample from an organ transplant recipient into one of two classes comprising liver transplant rejection and normal transplant function (TX). In some embodiments, a three-way classifier may classify a sample from a subject into one of three classes. A three-way classifier may classify a sample from an organ transplant recipient into one of three classes comprising AR, ADNR, and TX. In some embodiments, the classifier may work by applying two or more classifiers sequentially.

The methods, kits, and systems disclosed herein may comprise one or more algorithms or uses thereof. Algorithms such as those described in U.S. application Ser. No. 14/481,167, filed Sep. 9, 2014, may be used in the methods, kits, and systems disclosed herein. The one or more algorithms may be used to classify one or more samples from one or more subjects. The one or more algorithms may be applied to data from one or more samples. The data may comprise gene expression data. The data may comprise sequencing data. The data may comprise array hybridization data. Additionally, the classifiers described in U.S. application Ser. No. 14/481,167, filed Sep. 9, 2014, may be used in the methods, kits, and systems disclosed herein.

### Diagnosis, Prognosis and Monitoring

The methods provided herein can provide a value or other designation for a patient which indicates whether the aggregate measured expression levels in a patient is more like liver transplant patients with one of the graft injury phenotypes noted above (e.g., AR, ADNR, or TX). Such a value provides an indication that the patient either has or is at enhanced risk of developing a specific graft injury, or conversely does not have or is at reduced risk of having that specific graft injury phenotype. Risk is a relative term in which risk of one patient is compared with risk of other patients either qualitatively or quantitatively. For example, the value of one patient can be compared with a scale of values for a population of patients having undergone liver transplant to determine whether the patient's risk relative to that of other patients. In general, diagnosis is the determination of the present condition of a patient (e.g., presence or absence of a graft injury) and prognosis is developing future course of the patient (e.g., risk of developing liver transplant rejection or injury in the future or likelihood of improvement in response to treatment); however, the analyses contemplated by these terms may overlap or even be the same. For example, the methods of the disclosure alone do not necessarily distinguish between presence and enhanced risk of a liver transplant injury. However, these possibilities can be distinguished by additional testing.

In some embodiments, the methods, compositions, systems and kits described herein provide information to a medical practitioner that can be useful in making a therapeutic decision. Therapeutic decisions may include decisions to: continue with a particular therapy, modify a particular therapy, alter the dosage of a particular therapy, stop or terminate a particular therapy, alter the frequency of a therapy, introduce a new therapy, introduce a new therapy to be used in combination with a current therapy, or any combination of the above. In some embodiments, the results of diagnosing, predicting, or monitoring a condition of a transplant recipient may be useful for informing a therapeutic decision such as removal of the transplant. In some embodiments, the removal of the transplant can be an immediate removal. In some embodiments, the therapeutic decision can be a re-transplant. Other examples of therapeutic regimen can include a blood transfusion in instances where the transplant recipient is refractory to immunosuppressive or antibody therapy.

If a patient is indicated as having or being at enhanced risk of a liver transplant injury, the physician can subject the patient to additional testing including performing a liver biopsy, or performing other analyses such as examining whether there is an increase in bilirubin or liver enzyme levels, or both. Additionally or alternatively, the physician can change the treatment regime being administered to the patient. This includes administration of steroid boluses and the addition of other drugs to the maintenance therapy, or the administration of anti-lymphocyte antibodies in case of resistance to the primary line of therapy. In some embodiments, the change in treatment regime can include administering an additional or different drug to a patient, or administering a higher dosage or frequency of a drug already being administered to the patient. Many different drugs are available for treating rejection, such as immunosuppressive drugs used to treat transplant rejection, calcineurin inhibitors (e.g., cyclosporine, tacrolimus), mTOR inhibitors (e.g., sirolimus and everolimus), anti-proliferatives (e.g., azathioprine, mycophenolic acid), corticosteroids (e.g., prednisolone and hydrocortisone) and antibodies (e.g., basiliximab, daclizumab, Orthoclone, anti-thymocyte globulin and anti-lymphocyte globulin). In the case of HCV recurrence, the patients may be additionally administered drugs to counter the viral infection, e.g., interferons, ribavirin, and protease inhibitors.

Conversely, if the value or other designation of aggregate expression levels of a patient indicates the patient does not have or is at reduced risk of graft injury, the physician need not order further diagnostic procedures, particularly not invasive ones such as biopsy. Further, the physician can continue an existing treatment regime, or even decrease the dose or frequency of an administered drug.

In some methods of the disclosure, expression levels are determined at intervals in a particular patient (i.e., monitoring). Preferably, the monitoring is conducted by serial minimally-invasive tests such as blood draws; but, in some cases, the monitoring may also involve analyzing a liver biopsy, either histologically or by analyzing a molecular profile. The monitoring may occur at different intervals, for example the monitoring may be hourly, daily, weekly, monthly, yearly, or some other time period, such as twice a month, three times a month, every two months, every three months, etc.

Such methods can provide a series of values changing over time indicating whether the aggregate expression levels in a particular patient are more like the expression levels in patients undergoing a specific liver transplant rejection/injury or not undergoing the rejection/injury. Movement in value toward or away from the graft injury can provide an indication whether an existing immunosuppressive regime is working, whether the immunosuppressive regime should be changed, or whether a biopsy or increased monitoring by other markers rate should be performed.

The methods provided herein include administering a blood test (e.g., a test to detect acute rejection) to a transplant recipient who has already undergone a surveillance or protocol biopsy of the liver and received a biopsy result in the form of a histological analysis or a molecular profiling analysis. In some particular instances, the analysis of the liver biopsy (e.g., by histology or molecular profiling) may result in ambiguous, inconclusive, or borderline results. In such cases, a blood test provided herein may assist a caregiver with determining whether the transplant recipient has acute rejection or with interpreting the biopsy. In other cases the biopsy itself may be inconclusive or ambiguous, and in such cases the molecular analysis of the biopsy may be used in adjunct with the histology to confirm a diagnosis. In some instances, the analysis of the liver biopsy may yield a negative result. In such cases, the subject may receive a blood test provided herein in order to confirm the negative result, or to detect acute rejection or other transplant condition. In some cases, after receiving any type of biopsy result (e.g., negative result, ambiguous, inconclusive, borderline, positive), the patient may receive multiple, serial blood tests to monitor changes in molecular markers correlated with acute rejection.

The methods provided herein also include administering a biopsy test (e.g., histology or molecular profiling) to a transplant recipient who has received a molecular blood profiling test. For example, the transplant recipient may receive an ambiguous, inconclusive, or borderline result on a blood molecular profiling test. In such cases, the patient's healthcare worker may use the results of a liver biopsy test as a complement to the blood test to determine whether the subject is experiencing acute rejection. In another example, the transplant recipient may have received a positive result on a blood molecular profiling test, indicating that the transplant recipient has, or likely has, acute rejection, or even multiple positive results over time. In such cases, the patient's physician or other healthcare worker may decide to biopsy the patient's liver in order to detect liver transplant rejection. Such liver transplant rejection test may be a molecular profiling analysis of the patient's liver, as described herein. In some cases, a histological analysis of the liver biopsy may be performed instead of, or in addition to, the molecular analysis of the biopsy. In some cases, the physician may decide to wait a certain period of time after receiving the positive blood result to perform the biopsy test.

The methods provided herein may often provide early detection of liver transplant rejection and may help a patient to obtain early treatment such as receiving immunosuppressive therapy or increasing an existing immunosuppressive regimen. Such early treatment may enable the patient to avoid more serious consequences associated with acute rejection later in time, such as allograft loss. In some cases, such early treatments may be administered after the patient receives both a molecular profiling blood test and a biopsy analyzed either by molecular profiling or histologically.

### Drug Screening

The expression profiles associated with a liver transplant rejection/injury or lack thereof provided by the disclosure are useful in screening drugs, for example in clinical trials or in animal models of the injury. A clinical trial can be performed on a drug in similar fashion to the monitoring of an individual patient described above, except that drug is administered in parallel to a population of liver transplant patients, usually in comparison with a control population that is administered a placebo.

The changes in expression levels of genes can be analyzed in individual patients and across a treated or control population. Analysis at the level of an individual patient provides an indication of the overall status of the patient at the end of the trial (i.e., whether gene expression profile indicates presence or enhanced susceptibility to a liver transplant rejection/injury) and/or an indication whether that profile has changed toward or away from such indication in the course of the trial. Results for individual patients can be aggregated for a population allowing comparison between treated and control populations.

Similar trials can be performed in non-human animal models of chronic liver disease, e.g., the animal model described in Liu et al., Am. J. Physiol. Gastrointest Liver Physiol. 304:G449-68, 2013. With the animal models, the expression levels of genes detected are the species variants or homologs of the human genes referenced herein in whatever species of non-human animal on which tests are being conducted. Although the average or mean expression levels of human genes determined in human liver transplant patients undergoing or not undergoing a specific transplant rejection/injury are not necessarily directly comparable to those of homolog genes in an animal model, the human values can nevertheless be used to provide an indication whether a change in expression level of a non-human homolog is in a direction toward or away from an injury or susceptibility thereto. The expression profile of individual animals in a trial can provide an indication of the status of the animal at the end of the trial with respect to presence or susceptibility to the injury and/or change in such status during the trial. Results from individual animals can be aggregated across a population and treated and control populations compared. Average changes in the expression levels of genes can then be compared between the two populations.

### Computer Implemented Methods

Expression levels can be analyzed and associated with status of a subject (e.g., presence or susceptibility to a liver transplant injury) in a digital computer. Optionally, such a computer is directly linked to a scanner or the like receiving experimentally determined signals related to expression levels. Alternatively, expression levels can be input by other means. The computer can be programmed to convert raw signals into expression levels (absolute or relative), compare measured expression levels with one or more reference expression levels, or a scale of such values, as described above. The computer can also be programmed to assign values or other designations to expression levels based on the comparison with one or more reference expression levels, and to aggregate such values or designations for multiple genes in an expression profile. The computer can also be programmed to output a value or other designation providing an indication of presence or susceptibility to a liver transplant rejection or injury as well as any of the raw or intermediate data used in determining such a value or designation. The computer can also be used to run statistical tools and algorithms that test the data for patterns of expression that could be diagnostic or prognostic, as well as test for the validity and utility of gene signatures.

A typical computer (see U.S. Pat. No. 6,785,613 FIGS. 4 and 5) includes a bus which interconnects major subsystems such as a central processor, a system memory, an input/output controller, an external device such as a printer via a parallel port, a display screen via a display adapter, a serial port, a keyboard, a fixed disk drive and a floppy disk drive operative to receive a floppy disk. Many other devices can be connected such as a scanner via I/O controller, a mouse connected to serial port or a network interface. The computer contains computer readable media holding codes to allow the computer to perform a variety of functions. These functions include controlling automated apparatus, receiving input and delivering output as described above. The automated apparatus can include a robotic arm for delivering reagents for determining expression levels, as well as small vessels, e.g., microtiter wells for performing the expression analysis.

The methods, systems, kits and compositions provided herein may also be capable of generating and transmitting results through a computer network. Additionally, the computer programs, non-transitory computer-readable storage medium, web applications, mobile applications, stand-alone applications, web browser plug-ins, software modules, databases, and data transmissions described in U.S. application Ser. No. 14/481,167, filed Sep. 9, 2014, may be used in the methods, kits, and systems disclosed herein.

### EXAMPLES

Non-invasive biomarker profiles of acute rejection (AR) could significantly impact the management of liver transplant (LT) recipients. The aim of the studies disclosed herein was to discover and validate blood genomic signatures diagnostic and predictive of AR following LT. Peripheral blood was collected following LT for discovery (Northwestern University (NU) biorepository) and validation (NIAID CTOT-14 study). Blood gene expression profiling was paired with for-cause biopsies and characterized as AR or ADNR (acute dysfunction no rejection) based on central pathology reads. Samples from patients with longstanding stable graft function were used as controls (Transplant eXcellent—TX). CTOT-14 subjects had serial blood collections prior to AR, ADNR, and TX, as well as after AR treatment. NU cohort gene expression data (46 AR, 45 TX) were analyzed using random forest models to generate a classifier training set selected based on predictive performance. A 36-gene probe set (Table 3) distinguished AR vs. TX (AUC 0.92). The algorithm and 0.5 threshold were then locked and tested on the CTOT-14 validation cohort (14 AR, 50 TX), yielding an accuracy of 0.77, sensitivity 0.57, specificity 0.82, PPV 0.47, and NPV 0.87 for AR vs. TX. The probability score line slopes were positive preceding AR and different than the negative slopes preceding TX and non-AR (TX+ADNR) (p=<0.001). The slope became negative following treatment of AR. Ingenuity pathway analysis of AR genes mapped to hepatic toxicity and proliferation pathways. In conclusion, the following examples demonstrate a blood-based biomarker profile diagnostic for AR vs. non-AR that is also predictive prior to the onset of AR and abnormal liver function tests. Further studies will evaluate its utility in precision monitoring during immunosuppression modifications following LT.

### Example 1

### Methods

Cohorts and Subjects. Beginning in 2010, adult deceased or live donor LT recipients undergoing ‘for cause’ graft biopsies were enrolled into the NU biorepository study. Blood samples for biomarker studies were collected at the time of biopsy. In addition, samples were also collected from patients with longstanding normal LFTs following LT for non-viral etiologies. Samples from these patients were characterized as ‘Transplant eXcellent’ (TX) and were labeled ‘virtual biopsies’ as negative controls. Samples from NU biorepository subjects were used as the primary training set for biomarker discovery.

An independent external cohort of 186 adult deceased or live donor LT recipients was also enrolled into the multicenter observational CTOT-14 study between August 2012 and December 2015. Subjects were followed for 12-24 months. Recipients underwent ‘for cause’ biopsies for acute graft dysfunction per each participating site's standard of care. Blood samples were collected for biomarker studies at the time of biopsy and also serial blood samples at week 2, month 1, 2, 3, 6, 9, 12, 15, 18, 21, 24 following LT. Biomarker analyses were limited to month 12 as only a small percentage of subjects were followed to month 24. Samples from CTOT-14 subjects were used validate the molecular classifiers, algorithm and threshold derived from the NU discovery cohort. In addition, because of the serial collection of blood samples and the longitudinal information on the clinical course of these subjects, the ability of these validated classifiers to predict the onset of AR vs. non-AR and resolution of the AR phenotype was tested.

Inclusion and exclusion criteria for both cohorts were identical and consisted of male or female (negative pregnancy test within 6 weeks of enrollment in CTOT-14) LT recipients, age ≥18 years, ability to provide informed consent, recipient of first LT from either a deceased or living donor. Subjects who had received prior or multi-organ transplants or were HIV-infected were excluded from this study. While not excluded from overall enrollment, patients with hepatitis C virus (HCV) and active viremia were excluded from biomarker analysis, to avoid HCV as a confounder in gene expression and because of the diminishing clinical relevance of HCV infection in LT (30). Seropositive non-viremic recipients (i.e., sustained virological response to prior therapy) were included, however. Clinical care for both cohorts followed standard practice at each participating site. All NU and CTOT14 biopsy specimens were processed locally for routine histology and sent to the University of Pittsburgh for an independent, blinded central review. Rejection was scored using the Banff Rejection Activity Index (31). LT recipients with RAI score ≥3 were classified as AR. Other non-AR causes, such as steatohepatitis, cholestasis, ischemic or drug injury, or other etiology, were grouped together as acute dysfunction no rejection (ADNR). Clinical and histological data were also reviewed by a transplant hepatologist and a phenotype was designated for each liver biopsy and sample collected.

For the NU cohort, demographic, patient characteristics, laboratory, medication, and clinical course data before and after liver biopsy or virtual TX biopsy were collected from the Northwestern Medicine® Enterprise Data Warehouse, which is a single, comprehensive and integrated repository of all clinical and research data. For the CTOT14 cohort, similar data was collected serially in an electronic database MEDIDATA RAVE managed by the Data Coordinating Center (DCC—Rho® Federal Systems). The NU biorepository and CTOT-14 studies were both subject to IRB approval and informed consent was obtained from all patients. Oversight by the DCC included development of the study protocol, review of clinical site visits, classification of clinical phenotypes at the sample level, validation of analyses and manuscript review.

Clinical Phenotypes (CPs). Clinical, biochemical and liver/virtual biopsy criteria were used to define four CPs: AR, ADNR, TX and non-AR (combination of ADNR and TX). For the purpose of biomarker development, these CPs were confirmed by the DCC and CPs of validation samples were blinded to the biomarker team. Assignment of the CPs for both the discovery and validation cohorts utilized the following algorithms:

1. AR: ‘for cause biopsy’ for abnormal LFTs (acute change) per site criteria; central biopsy read consistent with AR.
2. ADNR: ‘for cause biopsy’ for abnormal LFTs (acute change) per site criteria; central biopsy read inconsistent with etiology other than AR.
3. TX: defined as normal LFTs at the time of ‘virtual biopsy’. For NU subjects, there was a requirement for LFTs 3 months before and 3 months after the ‘virtual biopsy’. Because all CTOT14 subjects had at least 12 months of follow-up with a collection at six months post-LT, subjects with normal LFTs were chosen at this middle time point with the requirement that >50% of their LFTs leading up this time point were also within normal limits. The same criteria were used to define ‘normal’ LFTs for both cohorts: Total Bilirubin (TB)<1.5 mg/dL and Direct Bilirubin <0.5 mg/dL, Alkaline Phosphatase (AP)<200 U/L, and Alanine Transaminase (ALT) <60 U/L (males)<36 U/L (females).
4. Non-AR: ADNR and TX (as defined above)

Pre-ADNR, -TX, and -non-AR samples were used as controls for pre-AR to ensure specificity for predicting AR vs. the other CPs. For both Pre-AR and Pre-ADNR, only the first CPs of either AR or ADNR were used for serial sample analyses. The following criteria were used as for the pre-analysis:

1. Pre-AR: ≥1 sample(s) collected prior to for cause liver biopsy demonstrating AR (as defined above); at least 2 of the 3 liver tests (TB, AP, ALT) were normal at the time of the predictive sample collection; samples collected when ALT>100 U/L were excluded from use, even if AP and TB were normal.
2. Pre-ADNR: identical criteria as pre-AR, except for-cause biopsy demonstrating ADNR (as defined above).
3. Pre-TX: identical criteria as pre-AR, except virtual biopsy met TX criteria (as defined above). 4. Pre-non-AR: pre-ADNR and pre-TX samples combined.

For post-diagnosis, the focus was on gene expression changes following treatment of AR. Frequency of collection of samples was not intensified after non-AR.

5. Post-AR: collection of blood samples in CTOT-14 subjects treated for AR per site protocol was requested every 2 weeks for 8 weeks. As these repeat collections were not strictly adhered to, all subjects with at least 1 sample collected following treatment that coincided with resolution of AR as defined by normal LFTs (defined above) were used for this analysis.

Biomarker development: All peripheral blood samples used for biomarker development were drawn directly into PAXgene (BD BioSciences, San Jose, Calif.) tubes and processed as previously described (32). Samples were then subjected to a workflow as previously also as previously described (33). In the NU discovery cohort, the top 5000 variables probes were selected based on coefficient of variance, and the most informative genes for prediction were identified using random forests (RF) and Gini importance providing a relative ranking of the classifier features (34). Features selection was performed on these probes using a random-forest based algorithm with 5000 trees. The variable selection algorithm uses out of bag error as minimization criterion, carry out variable elimination from random forest, by successively eliminating the least important variables (35). An optimal performance threshold was selected favoring NPV over PPV, and the model and threshold were then locked for validation using an independent external CTOT-14 cohort. Diagnostic performance metrics included predictive accuracy, sensitivity, specificity, negative predictive value (NPV), positive predictive value (PPV), and area under the curve (AUC). The same locked model and threshold were also used on pre-AR, -TX and -non-AR samples as well as post-treatment of AR samples. As each subject underwent sampling multiple times at different time points, a linear mixed effect model with random intercept was used to estimate the pre-biopsy (or virtual TX biopsy) slope for each phenotype to account for within patient correlation. Data were first stratified by phenotypes and coefficients were estimated and compared via linear mixed effect model. Another linear mixed effect model with both random slope and random intercept was fitted to compare the slopes of pre-biopsy and post-biopsy for only the AR patients. Analysis was performed using R version 3.5.1 via RStudio.

Probes from the final locked models were then fed to Ingenuity Core Analysis (Qiagen, Inc., Hilden, Germany) that provides information about enriched pathways and allows comparison to findings from the scientific literature, including information associated with cardiotoxicity, nephrotoxicity, and hepatotoxicity (36). Enriched pathways were selected based on Fisher's exact test p-values such that a value of <=0.05 was considered statistically significant.

Gene Microarrays: Whole blood was collected in PAXgene PAXgene Blood RNA (IVD) tubes (Qiagen, Valencia, Calif.). Total RNA was extracted from PAXgene tubes using PAXgene Blood micro RNA (miRNA) reagents on the QIAcube instrument (Qiagen). Total RNA yield and concentration were determined using the Nanodrop 8000 (Thermo Fisher Scientific). Blood samples involved an additional globin RNA reduction step using the Ambion GLOBINclear Human kit (Thermo Fisher Scientific). The Affymetrix 3′ IVT (in vitro transcript) PLUS labeling system was used for in vitro transcription and labeling reactions (3′ IVT) on 200 ng of globin-reduced RNA (Affymetrix, Santa Clara, Calif.). Array hybridization washing, staining and scanning was done using standard manufacturer's workflow (Affymetrix, Santa Clara, Calif.).

Statistical Analysis: Baseline demographics and clinical characteristics were compared between the groups via standard categorical and continuous variable statistical tests. Microarray signals were normalized with robust multi-array analysis. For detecting the diagnostic signatures, first top 500 variables probes were selected based on coefficient of variance. Then, the most informative genes for prediction were identified using random forest (RF) methodology. The RF classification algorithm is an ensemble learning method that operates by constructing a user defined set of decision trees for training a model and then outputs the class that is the mode of the classes from the many trees. Random decision forests are used to correct for individual decision trees' habit of overfitting to the training set. Additionally, during the classifier selection stages, RF performs an implicit feature selection, using a small subset of “strong/significant/important variables” leading to its superior performance, specifically on high dimensional data. This implicit feature selection can be visualized by the “Gini importance” and can be used as a general indicator of feature relevance and an importance score can be derived, which provides a relative ranking of the classifier features. The variable selection algorithm uses out of bag error as minimization criterion, carry out variable elimination from random forest, by successively eliminating the least important variables. Diagnostic performance was based on retrospective prediction of known clinical phenotype. The NU cohort served as the diagnostic discovery group (AR vs. TX diagnosis), which generated a locked model at an optimal performance threshold and then validated on the CTOT14 diagnostic cohort. Predictive accuracy was calculated using the formula (TP+TN)/(TP+FP+FN+TN); (TP—True Positive, TN—True Negative, FP—False Positive, FN—False Negative). Diagnostic metrics included sensitivity, specificity, and area under the curve (AUC). This locked model was then tested on the CTOT14 serial sample collections to evaluate their prediction of the phenotypes, as well as response to treatment of AR. As each patient was measured multiple times at different time points, a linear mixed effect model with random intercept was used to estimate the pre-biopsy (or virtual TX BX) slope for each phenotype and account for within patient correlation. Data were first stratified by phenotypes (AR, TX, and non-AR: TX+ADNR combined) and coefficients were estimated and compared via linear mixed effect model. Another linear mixed effect model with both random slope and random intercept was fitted to compare the slopes of pre-biopsy and post-biopsy for only the AR patients. Days from biopsy was converted into months to adjust for the scale of the parameter estimate. Analysis was performed using R version 3.5.1 via RStudio. Probes from the final locked models were then fed to Ingenuity Core Analysis that provides information about enriched pathways and allows comparison to findings from the scientific literature, including information associated with cardiotoxicity, nephrotoxicity, and hepatotoxicity. Enriched pathways were selected based on Fisher's exact test p-values such that a value of <=0.05 was considered statistically significant.

### Results

Summary: Gene expression data from the NU cohort (46 AR, 45 TX) samples were analyzed using random forest models to generate a training set of molecular classifiers that were selected based on predictive performance. A 36 gene probe set was selected for AR vs TX (AUC 0.92). The algorithm and threshold were then locked and tested on the CTOT-14 validation cohort (14 AR, 28 ADNR, 50 TX samples), yielding an overall accuracy of 0.81, sensitivity 0.5, specificity 0.9, PPV 0.58, and NPV 0.87 for AR vs. TX. The probability scores for serial pre- and post-samples were then analyzed using the locked threshold; the slopes of the probability scores were positive preceding AR but negative preceding TX (p=0.00016) and non-AR (TX+ADNR; p=0.0015). Following AR treatment, the slope turned negative and the majority of the probability scores were classified as TX according to the locked threshold. Ingenuity pathway analysis of differentially expressed AR genes used to populate the RF models mapped to hepatic proliferation/hyperplasia pathways.

NU biorepository: A total of 91 samples were used for biomarker discovery—46 AR and 45 TX as ‘virtual biopsies’. 38 ADNR biopsies were also collected, for use in a comparative diagnostic analysis and the preceding sample analysis for the non-AR phenotype (TX+ADNR). The overall cohort was 50.8±13.0 years old at transplant, 53.5% male, 72.8% Caucasian, 23.2% Non-Alcoholic Fatty Liver/Cryptogenic, 19.4% Alcoholic, 30.2% Immune-Mediated (primary biliary cirrhosis, primary sclerosing cholangitis, autoimmune hepatitis), 3.1% HCV non-viremic, 24% other cause of primary liver disease. Between the phenotypes, the only differences were that AR subjects were younger, closer to transplant, and as expected had LFT differences (Table 1).

TABLE 1
NU AND CTOT14 Phenotypes - Patient Characteristics
TABLE 1: NU AND CTOT14 PHENOTYPES - PATIENT CHARACTERISTICS

| | NU | | NU | | NU | | | CTOT14 | | CTOT14 | | CTOT14 | | |
|---|---|---|---|---|---|---|---|---|---|---|---|---|---|---|
| | AR | | ADNR | | TX | | P- | AR | | ADNR | | TX | | P- |
| | (N = 46) | | (N = 38) | | (N = 45) | | VALUE¹ | (N = 14) | | (N = 28) | | (N = 50) | | VALUE¹ |
| NUMBER OF SUBJECTS | 46 | | 38 | | 45 | | | 13 | | 20 | | 45 | | |
| AGE AT TRANSPLANT | 46 ± 17.4 | | 51.6 ± 13.0 | | 54.9 ± 13.0 | | 0.016 | 57 ± 12.9 | | 52 ± 13.7 | | 54 ± 11.4 | | 0.489 |
| (YEARS) | | | | | | | | | | | | | | |
| CAUCASIAN | 30 | (65%) | 28 | (74%) | 36 | (80%) | 0.276 | 12 | (92%) | 17 | (85%) | 38 | (84%) | 0.769 |
| RACE (%) | | | | | | | | | | | | | | |
| MALE SEX (%) | 18 | (39%) | 20 | (53%) | 14 | (31%) | 0.143 | 8 | (62%) | 12 | (60%) | 27 | (60%) | 0.995 |
| PRIMARY LIVER | | | | | | | | | | | | | | |
| DIAGNOSIS (%) | | | | | | | | | | | | | | |
| HEPATITIS C | 1 | (2%) | 1 | (3%) | 2 | (4%) | 0.248 | 4 | (31%) | 2 | (10%) | 0 | | 0.065 |
| (NON-VIREMIC) | | | | | | | | | | | | | | |
| ALCOHOL | 6 | (13%) | 7 | (18%) | 12 | (27%) | | 2 | (15%) | 4 | (20%) | 12 | (27%) | |
| NON-ALCOHOLIC | 7 | (15%) | 6 | (16%) | 17 | (38%) | | 3 | (23%) | 8 | (40%) | 18 | (40%) | |
| FATTY LIVER OR | | | | | | | | | | | | | | |
| CRYPTOGENIC | | | | | | | | | | | | | | |
| IMMUNE-MEDIATED | 16 | (35%) | 16 | (42%) | 7 | (16%) | | 1 | (8%) | 2 | (10%) | 7 | (16%) | |
| (PSC, AIH, PBC) | | | | | | | | | | | | | | |
| OTHER | 16 | (35%) | 8 | (21%) | 7 | (16%) | | 3 | (23%) | 4 | (20%) | 8 | (18%) | |
| MONTHS FROM LT² | 27.6 ± 34.8 | | 60 ± 57.6 | | 37 ± 37.2 | | 0.003 | 8 ± 7.1 | | 6 ± 5.4 | | 7 ± 2.0 | | 0.279 |
| IMMUNOSUPPRESSION² | | | | | | | | | | | | | | |
| CNI THERAPY | 42 | (91%) | 31 | (82%) | 41 | (91%) | 0.357 | 12 | (86%) | 22 | (79%) | 50 | (100%) | 0.004 |
| MMF OR MPA | 28 | (61%) | 17 | (45%) | 26 | (56%) | 0.322 | 9 | (64%) | 10 | (36%) | 36 | (72%) | 0.007 |
| OTHER (SRL, EVL, AZA) | 6 | (13%) | 2 | (5%) | 2 | (5%) | 0.292 | 3 | (21%) | 3 | (11%) | 2 | (4%) | 0.114 |
| PREDNISONE | 22 | (48%) | 17 | (45%) | 20 | (46%) | 0.95 | 7 | (50%) | 18 | (64%) | 37 | (74%) | 0.222 |

| LABORATORY | | | | | | | | | | |
|---|---|---|---|---|---|---|---|---|---|---|
| VALUES² | | | | | | | | | | |
| ALT (U/L) | 322.2 ± 260.5 | | 96.6 ± 75.9 | 21.4 ± 11.3 | <0.001 | 219.9 ± 265.14 | | 212.5 ± 251.80 | 21.6 ± 8.96 | <0.001 |
| ALKALINE | 332.3 ± 321.2 | | 275 ± 206 | 79.3 ± 30.9 | <0.001 | 269.3 ± 239.33 | | 281.6 ± 233.40 | 96.6 ± 37.35 | <0.001 |
| PHOSPHATASE (U/L) | | | | | | | | | | |
| TOTAL BILIRUBIN | 4.1 ± 9.0 | | 6 ± 22.3 | 0.6 ± 0.2 | <0.001 | 4.1 ± 8.28 | | 3.5 ± 4.49 | 0.6 ± 0.31 | 0.001 |
| (MG/DL) | | | | | | | | | | |
| CREATININE (MG/DL) | 1.2 ± 0.6 | | 1.3 ± 0.4 | 1.3 ± 0.4 | 0.529 | 1.3 ± 0.44 | | 1.5 ± 0.87 | 1.2 ± 0.31 | 0.056 |
| REJECTION | | | | | | | | | | |
| CHARACTERISTICS² | | | | | | | | | | |
| MILD (RAI 3-4) | 19 | (41%) | — | — | — | 7 | (50%) | — | — | — |
| MODERATE -SEVERE | 27 | (59%) | — | — | — | 7 | (50%) | — | — | — |
| (RAI 5-9) | | | | | | | | | | |

¹p-value reported using the overall F-test from ANOVA for continuous variables and the CMH test of general association for categorical variables.
²Reported characteristic corresponds to the time of diagnostic sample. For TX subjects in CTOT14, immunosuppression data reported using the month 3 time point.

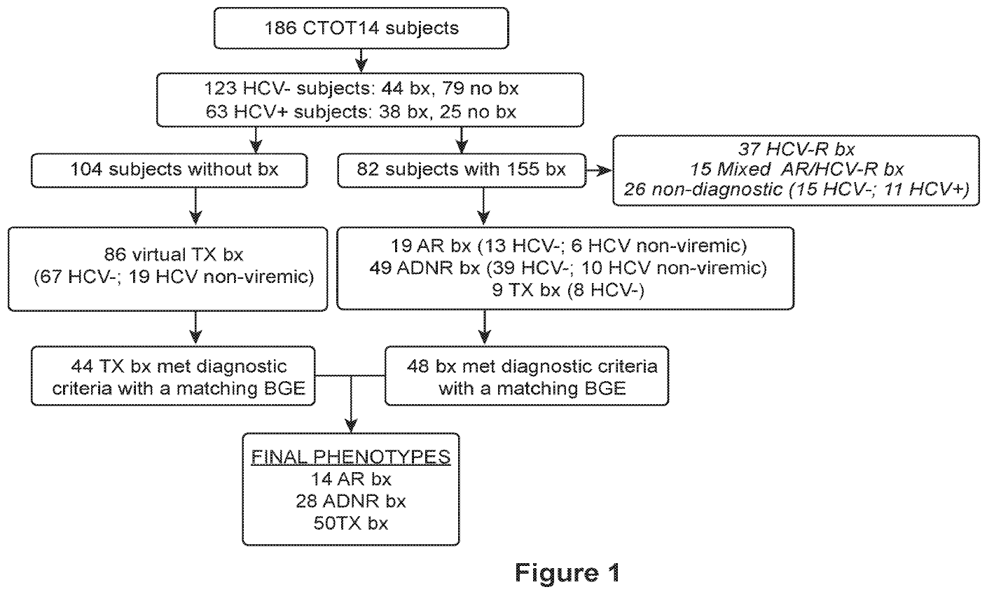

CTOT14: 186 LT recipients were enrolled. However, the final analysis included 78 patients who had 92 distinct phenotypes (14 AR biopsies, 28 ADNR biopsies, 50 TX BX) with accompanying samples (FIG. 1). The overall cohort was 54±11.7 years old at transplant, 60% male, 87% Caucasian, 36% Non-Alcoholic Fatty Liver/Cryptogenic, 24% Alcoholic, 13% Immune-Mediated, 8% HCV non-viremic, 19% other cause of primary liver disease. Between the phenotypes, there were differences in CNI and mycophenolic acid use, and both AR and ADNR had higher LFTs (Table 1).

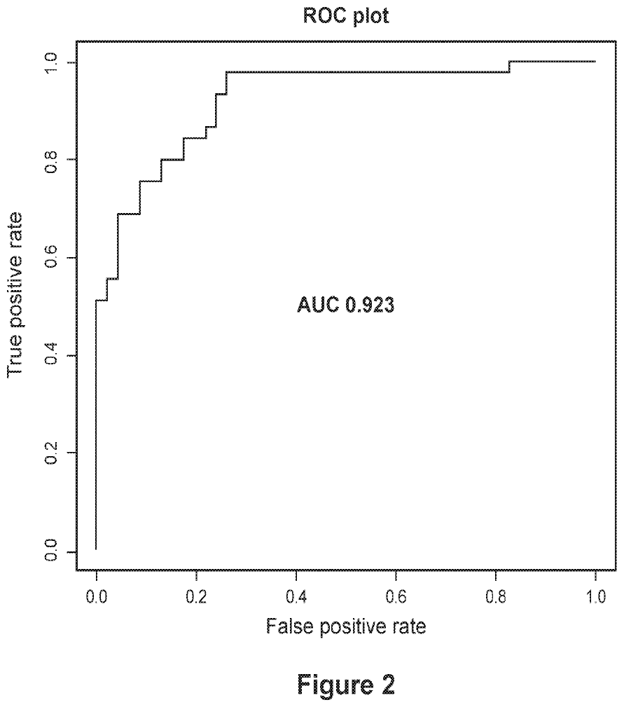

Whole blood samples at the time of biopsy or TX BX time point were analyzed by microarray. Selected probes were then used to generate the final RF model with 36-gene probes to differentiate AR vs. TX phenotypes. This resulted in an overall high predictive accuracy (AUC 0.923) for AR vs. TX at diagnosis (FIG. 2). At a threshold of 0.5 (Table 2), the accuracy was 0.82, sensitivity 0.83, specificity 0.82, prevalence-adjusted PPV 0.57, and prevalence-adjusted NPV 0.94. The prevalence was adjusted using the CTOT14 CP prevalence, as NU was not a true prevalent population with single time point samples being collected at any time post-LT.

TABLE 2
NU Discovery
AR vs. TX

| | | | | | | | | | | | | Prevalence- | Prevalence- |
|---|---|---|---|---|---|---|---|---|---|---|---|---|---|
| | | | | | | | | | | | | adjusted | adjusted |
| Threshold | Total P | Total N | True P | False P | True N | False N | accuracy | sensitivity | specificity | PPV | NPV | PPV | MPV |
| 0.3 | 46 | 45 | 44 | 16 | 29 | 2 | 0.80 | 0.96 | 0.64 | 0.73 | 0.94 | 0.43 | 0.98 |
| 0.32 | 46 | 45 | 44 | 15 | 30 | 2 | 0.81 | 0.96 | 0.67 | 0.75 | 0.94 | 0.45 | 0.98 |
| 0.34 | 46 | 45 | 44 | 14 | 31 | 2 | 0.82 | 0.96 | 0.69 | 0.76 | 0.94 | 0.46 | 0.98 |
| 0.36 | 46 | 45 | 43 | 13 | 32 | 3 | 0.82 | 0.93 | 0.71 | 0.77 | 0.91 | 0.48 | 0.97 |
| 0.38 | 46 | 45 | 42 | 13 | 32 | 4 | 0.81 | 0.91 | 0.71 | 0.76 | 0.89 | 0.47 | 0.97 |
| 0.4 | 46 | 45 | 42 | 13 | 32 | 4 | 0 81 | 0.91 | 0.71 | 0.76 | 0.89 | 0.47 | 0.97 |
| 0.42 | 46 | 45 | 42 | 13 | 32 | 4 | 0.81 | 0.91 | 0.71 | 0.76 | 0.89 | 0.47 | 0.97 |
| 0.44 | 46 | 45 | 40 | 11 | 34 | 6 | 0.81 | 0.87 | 0.76 | 0.78 | 0.85 | 0.50 | 0.95 |
| 0.46 | 46 | 45 | 40 | 11 | 34 | 6 | 0.81 | 0.87 | 0.76 | 0.78 | 0.85 | 0.50 | 0.95 |
| 0.48 | 46 | 45 | 39 | 10 | 35 | 7 | 0.81 | 0.85 | 0.78 | 0.80 | 0.83 | 0.52 | 0.95 |
| 0.5 | 46 | 45 | 38 | 8 | 37 | 8 | 0.82 | 0.83 | 0.82 | 0.83 | 0.82 | 0.57 | 0.94 |
| 0.52 | 46 | 45 | 38 | 7 | 38 | 8 | 0.84 | 0.83 | 0.84 | 0.84 | 0.83 | 0.60 | 0.95 |
| 0.54 | 46 | 45 | 35 | 7 | 38 | 11 | 0.80 | 0.76 | 0.84 | 0.83 | 0.78 | 0.58 | 0.93 |
| 0.56 | 46 | 45 | 36 | 6 | 39 | 10 | 0.82 | 0.78 | 0.87 | 0.86 | 0.80 | 0 62 | 0.93 |
| 0.58 | 46 | 45 | 35 | 5 | 40 | 11 | 0.82 | 0.76 | 0.89 | 0.88 | 0.78 | 0.66 | 0.93 |
| 0.6 | 46 | 45 | 35 | 2 | 43 | 11 | 0.86 | 0.76 | 0.96 | 0.95 | 0.80 | 0.83 | 0.93 |
| 0.62 | 46 | 45 | 34 | 1 | 44 | 12 | 0.86 | 0.74 | 0.98 | 0.97 | 0.79 | 0.90 | 0.93 |
| 0.64 | 46 | 45 | 34 | 2 | 43 | 12 | 0.85 | 0.74 | 0.96 | 0.94 | 0.78 | 0.82 | 0.93 |
| 0.66 | 46 | 45 | 33 | 1 | 44 | 13 | 0.85 | 0.72 | 0.98 | 0.97 | 0.77 | 0.90 | 0.93 |
| 0.68 | 46 | 45 | 32 | 1 | 44 | 14 | 0.84 | 0.70 | 0.98 | 0.97 | 0.76 | 0.90 | 0.92 |
| 0.7 | 46 | 45 | 30 | 1 | 44 | 16 | 0.81 | 0.65 | 0.98 | 0.97 | 0.73 | 0.89 | 0.91 |

Using the selected probability threshold from the discovery cohort above, the locked NU 36-gene probe model (AR vs. TX) was then applied to the CTOT-14 cohort for validation. At the locked NU threshold of 0.5, the accuracy was 0.77, sensitivity 0.57, specificity 0.82, PPV 0.47, and NPV 0.87. When the 28 CTOT-14 ADNR diagnostic samples were analyzed on the same locked model and threshold for AR vs. TX, the majority (n=19; 68%) classified as TX and remaining as AR (n=9, 32%).

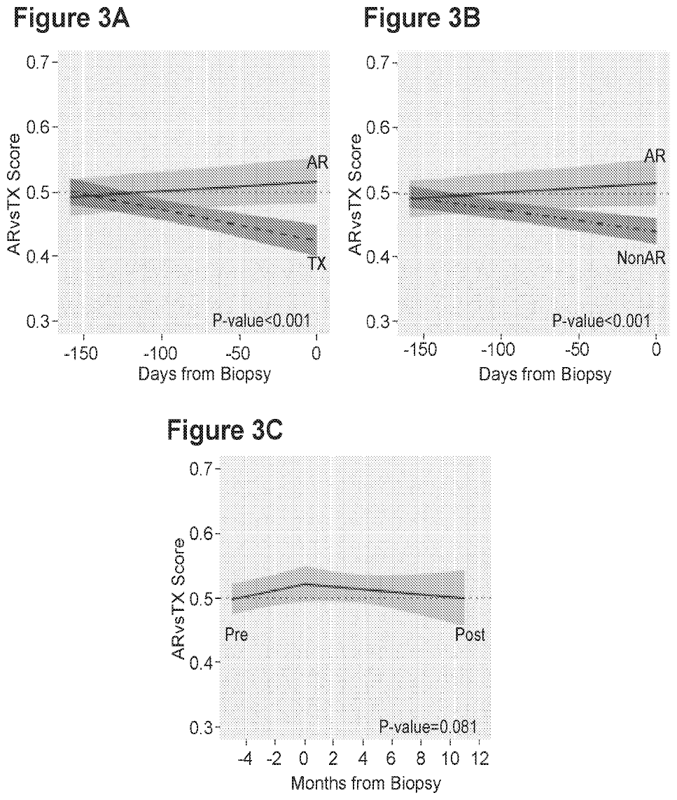

To test the predictive ability of the classifiers, 33 CTOT-14 serial samples collected in the weeks prior to 12 AR biopsies, 24 prior to 11 ADNR biopsies, and 155 samples collected prior to 47 TX virtual biopsies were used. To compare changes in gene expression over time prior to diagnosis, a linear mixed model analysis was performed comparing the slopes of each line of scores between the phenotypes. This showed a difference between the directions of the line slopes prior to AR (positive) vs. TX (negative) (FIG. 3a). When TX and ADNR was combined into a non-AR category to further distinguish AR patients' trajectory from the overall cohort, AR was also different in line slope direction than non-AR (FIG. 3b). The slopes for each analysis were also statistically different from each other in the mixed model (AR vs. TX, p<0.001; AR vs. non-AR, p<0.001), with the decline in slope prior to TX and non-AR being steeper over time than the incline prior to AR.

A similar analysis was performed for 53 samples following treatment of the 12 AR cases. Of note, all patients responded to treatment defined by normalization of LFTs. This analysis showed that the slope following treatment of AR turned negative, although not statistically different than pre-AR (FIG. 3c; p=0.081), suggesting that the increase in slope prior to AR was similar to the decrease in slope with treatment.

To evaluate the biological relevance of the probes in the AR vs. TX RF model, the 36 probes were tested using Ingenuity's pathway analysis (36). Many genes mapped to hepatic proliferation and inflammatory pathways—upregulated in AR and downregulated in TX. Tox analysis indicated that the highest percentage (38%) of genes from the locked model have been previously reported to be involved in Hepatotoxicity (Liver hyperplasia/hyper-proliferation). Principal component analysis demonstrated differential clustering of genes in AR vs. TX. Results of 3D-PCA score-plots showed a comparison of AR vs. TX NU cohort based on the top discriminative probes.

### DISCUSSION

A blood-based molecular biomarker has been developed and disclosed herein that correlates with the presence of absence of AR on paired graft biopsies following liver transplantation. This biologically relevant gene expression classifier model was optimized for NPV, and using a locked threshold, was validated in an independent, multi-center cohort. The data also show that the biomarker can help predict the onset of AR, in preceding samples in the setting of normal LFTs, differentially from those that precede non-AR. The biomarker also correlated with resolution of AR. The strength in having a model that performs well with serial assessments lies in the potential to use them sequentially in practice to proactively monitor for immune activation during interventions, such as IS minimization or withdrawal. In addition, the AR genes in the model disclosed herein mapped to pathways of hepatotoxicity and hyper-proliferation, which provides mechanistic support by linking LT rejection with genes of liver injury and regeneration.

In a recent publication, the development and clinical validity of a similar biomarker was described for subclinical rejection in kidney transplant recipients designed to detect silent rejection in patients with stable graft function (33). In that setting, a biomarker-informed approach was proposed as an alternative to the routine use of surveillance biopsies in an attempt to improve transplant outcomes while reducing the need for invasive biopsies. While surveillance biopsies are not used in LT recipients for the routine management of IS, there is a great need for non-invasive monitoring of patients undergoing IS minimization or withdrawal, as these protocols are becoming increasingly more common (3-11). While a number of exciting developments in this direction have been reported in kidney transplant and other organs (28, 32, 33, 37-45), there is a paucity of literature on similar developments in the LT literature. One of the main reasons for this might be the fact that chronic rejection in the absence of appropriate medication adherence is rare following LT in contrast with other organ transplants, and therefore there is less incentive to develop biomarkers to detect sub-clinical rejection. On the other hand, as IS minimization strategies continue to evolve and be of great interest, especially those that involve early CNI avoidance or withdrawal, there is a need to know whether or when an immune-quiescent state turns into immune-activation prior to the onset of biochemical graft dysfunction.

Another putative reason for the lack of progress in this area for LT may be related to difficulties inherent to the development of predictive biomarkers. A number of investigators have studied the use of genetic polymorphisms in the donor and recipient to predict the risk of rejection (17, 20, 21, 23, 24, 46-48). Others have explored circulating microRNA, peripheral blood lymphocytes, and complement proteins as being increased in acute rejection in LT, with some being predictive before events (15, 16, 18, 22, 28). In a pilot study, diagnostic proteoform signatures of acute rejection were identified in circulating immune cells, using an emerging “top down” proteomics methodology (49). However, the major limitation of these studies has been small sample sizes, lack of validation cohorts and statistical rigor, and tests that are less appropriate for serial monitoring in patient management. The stringent criteria needed for biomarker development were reviewed and strictly adhered to in designing the current study (50). As a result, the present disclosure provides the first gene expression biomarker that can be used to non-invasively monitor patients undergoing IS modifications, such as minimization or withdrawal studies.

In addition to peripheral blood, there has also been interest in liver graft-derived biomarkers that has historically focused on distinguishing histological injury due to HCV versus AR (25-27, 29). Given the advent of oral antiviral therapy for HCV, most patients are cured of HCV, lowering the importance of this issue in clinical practice. Interestingly, a recent study identified mRNA expression differences in blood and liver tissue in LT recipients developing AR with late IS withdrawal (29), and another study showed that graft mRNA expression was predictive of tolerance (51). While it is recognized that tissue biomarkers may have a role in tolerance prediction, they are only applicable to a small subset who are candidates for research-based tolerance studies. Blood-based tests as disclosed herein are more useful and practical for serial monitoring during routine IS modifications in the overall LT population.

Thus, Example 1 demonstrates a non-invasive biomarker profile diagnostic for AR vs. TX. This molecular profile was also predictive of the onset of AR, in the context of normal liver function tests pre-AR, and returns to TX following treatment.

### REFERENCES

1. Wiesner R H, Batts K P, Krom R A. Evolving concepts in the diagnosis, pathogenesis, and treatment of chronic hepatic allograft rejection. Liver Transpl Surg 1999; 5:388-400.
2. Watt K D, Pedersen R A, Kremers W K, Heimbach J K, Charlton M R. Evolution of causes and risk factors for mortality post-liver transplant: results of the NIDDK long-term follow-up study. American journal of transplantation: official journal of the American Society of Transplantation and the American Society of Transplant Surgeons 2010; 10:1420-1427.
3. Levitsky J, O'Leary J G, Asrani S, Sharma P, Fung J, Wiseman A, Niemann C U. Protecting the Kidney in Liver Transplant Recipients: Practice-Based Recommendations From the American Society of Transplantation Liver and Intestine Community of Practice. American journal of transplantation: official journal of the American Society of Transplantation and the American Society of Transplant Surgeons 2016; 16:2532-2544.
4. Levitsky J, Feng S. Tolerance in clinical liver transplantation. Hum Immunol 2018; 79:283-287.
5. Benitez C, Londono M C, Miguel R, Manzia T M, Abraldes J G, Lozano J J, Martinez-Llordella M, et al. Prospective multicenter clinical trial of immunosuppressive drug withdrawal in stable adult liver transplant recipients. Hepatology 2013; 58:1824-1835.
6. Feng S, Ekong U D, Lobritto S J, Demetris A J, Roberts J P, Rosenthal P, Alonso E M, et al. Complete immunosuppression withdrawal and subsequent allograft function among pediatric recipients of parental living donor liver transplants. JAMA 2012; 307:283-293.
7. De Simone P, Nevens F, De Carlis L, Metselaar H J, Beckebaum S, Saliba F, Jonas S, et al. Everolimus with reduced tacrolimus improves renal function in de novo liver transplant recipients: a randomized controlled trial. Am J Transplant 2012; 12:3008-3020.
8. Teperman L, Moonka D, Sebastian A, Sher L, Marotta P, Marsh C, Koneru B, et al. Calcineurin inhibitor-free mycophenolate mofetil/sirolimus maintenance in liver transplantation: the randomized spare-the-nephron trial. Liver Transpl 2013; 19:675-689.
9. Shaked A, DesMarais M R, Kopetskie H, Feng S, Punch J D, Levitsky J, Reyes J, et al. Outcomes of immunosuppression minimization and withdrawal early after liver transplantation. Am J Transplant 2019; 19:1397-1409.
10. Banff Working Group on Liver Allograft P. Importance of liver biopsy findings in immunosuppression management: biopsy monitoring and working criteria for patients with operational tolerance. Liver Transpl 2012; 18:1154-1170.
11. Demetris A J, Isse K. Tissue biopsy monitoring of operational tolerance in liver allograft recipients. Curr Opin Organ Transplant 2013; 18:345-353.
12. Kowalski R J, Post D R, Mannon R B, Sebastian A, Wright H I, Sigle G, Burdick J, et al. Assessing relative risks of infection and rejection: a meta-analysis using an immune function assay. Transplantation 2006; 82:663-668.
13. Xue F, Zhang J, Han L, Li Q, Xu N, Zhou T, Xi Z, et al. Immune cell functional assay in monitoring of adult liver transplantation recipients with infection. Transplantation 2010; 89:620-626.
14. Cabrera R, Ararat M, Soldevila-Pico C, Dixon L, Pan J J, Firpi R, Machicao V, et al. Using an immune functional assay to differentiate acute cellular rejection from recurrent hepatitis C in liver transplant patients. Liver Transpl 2009; 15:216-222.
15. Fan H, Li L X, Han D D, Kou J T, Li P, He Q. Increase of peripheral Th17 lymphocytes during acute cellular rejection in liver transplant recipients. Hepatobiliary Pancreat Dis Int 2012; 11:606-611
16. Farid W R, Pan Q, van der Meer A J, de Ruiter P E, Ramakrishnaiah V, de Jonge J, Kwekkeboom J, et al. Hepatocyte-derived microRNAs as serum biomarkers of hepatic injury and rejection after liver transplantation. Liver Transpl 2012; 18:290-297.
17. Gomez-Mateo J, Mann L, Lopez-Alvarez M R, Moya-Quiles M R, Miras M, Marin-Moreno I, Botella C, et al. TGF-beta1 gene polymorphism in liver graft recipients. Transpl Immunol 2006; 17:55-57.
18. Joshi D, Salehi S, Brereton H, Arno M, Quaglia A, Heaton N, O'Grady J, et al. Distinct microRNA profiles are associated with the severity of hepatitis C virus recurrence and acute cellular rejection after liver transplantation. Liver Transpl 2013; 19:383-394.
19. Kamei H, Masuda S, Nakamura T, Oike F, Takada Y, Hamajima N. Association of transporter associated with antigen processing (TAP) gene polymorphisms in donors with acute cellular rejection in living donor liver transplantation. J Gastrointestin Liver Dis 2013; 22:167-171.
20. Karimi M H, Daneshmandi S, Pourfathollah A A, Geramizadeh B, Malekhosseini S A, Nikeghbalian S, Yaghobi R, et al. Association of IL-6 promoter and IFN-gamma gene polymorphisms with acute rejection of liver transplantation. Mol Biol Rep 2011; 38:4437-4443.
21. Li H, Xie H Y, Zhou L, Feng X W, Wang W L, Liang T B, Zhang M, et al. Copy number variation in CCL3L1 gene is associated with susceptibility to acute rejection in patients after liver transplantation. Clin Transplant 2012; 26:314-321.
22. Massoud O, Heimbach J, Viker K, Krishnan A, Poterucha J, Sanchez W, Watt K, et al. Noninvasive diagnosis of acute cellular rejection in liver transplant recipients: a proteomic signature validated by enzyme-linked immunosorbent assay. Liver Transpl 2011; 17:723-732.
23. Moya-Quiles M R, Alvarez R, Miras M, Gomez-Mateo J, Lopez-Alvarez M R, Marin-Moreno I, Martinez-Barba E, et al. Impact of recipient HLA-C in liver transplant: a protective effect of HLA-Cw*07 on acute rejection. Hum Immunol 2007; 68:51-58.
24. Sindhi R, Higgs B W, Weeks D E, Ashokkumar C, Jaffe R, Kim C, Wilson P, et al. Genetic variants in major histocompatibility complex-linked genes associate with pediatric liver transplant rejection. Gastroenterology 2008; 135:830-839, 839 e831-810.
25. Asaoka T, Kato T, Marubashi S, Dono K, Hama N, Takahashi H, Kobayashi S, et al. Differential transcriptome patterns for acute cellular rejection in recipients with recurrent hepatitis C after liver transplantation. Liver Transpl 2009; 15:1738-1749.
26. Gehrau R, Maluf D, Archer K, Stravitz R, Suh J, Le N, Mas V. Molecular pathways differentiate hepatitis C virus (HCV) recurrence from acute cellular rejection in HCV liver recipients. Mol Med 2011; 17:824-833.
27. Sreekumar R, Rasmussen D L, Wiesner R H, Charlton M R. Differential allograft gene expression in acute cellular rejection and recurrence of hepatitis C after liver transplantation. Liver Transpl 2002; 8:814-821.
28. Shaked A, Chang B L, Barnes M R, Sayre P, Li Y R, Asare S, DesMarais M, et al. An ectopically expressed serum miRNA signature is prognostic, diagnostic, and biologically related to liver allograft rejection. Hepatology 2017; 65:269-280.
29. Bonaccorsi-Riani E, Pennycuick A, Londono M C, Lozano J J, Benitez C, Sawitzki B, Martinez-Picola M, et al. Molecular Characterization of Acute Cellular Rejection Occurring During Intentional Immunosuppression Withdrawal in Liver Transplantation. Am J Transplant 2016; 16:484-496.
30. Ishak K, Baptista A, Bianchi L, Callea F, De Groote J, Gudat F, Denk H, et al. Histological grading and staging of chronic hepatitis. J Hepatol 1995; 22:696-699.
31. Banff schema for grading liver allograft rejection: an international consensus document. Hepatology 1997; 25:658-663.
32. Kurian S M, Williams A N, Gelbart T, Campbell D, Mondala T S, Head S R, Horvath S, et al. Molecular classifiers for acute kidney transplant rejection in peripheral blood by whole genome gene expression profiling. Am J Transplant 2014; 14:1164-1172.
33. Friedewald J J, Kurian S M, Heilman R L, Whisenant T C, Poggio E D, Marsh C, Baliga P, et al. Development and clinical validity of a novel blood-based molecular biomarker for subclinical acute rejection following kidney transplant. Am J Transplant 2019; 19:98-109.
34. Breiman L. Machine Learning. 45 2001; 5.
35. Diaz-Uriarte R, Alvarez de Andres S. Gene selection and classification of microarray data using random forest. BMC Bioinformatics 2006; 7:3.
36. QIAGEN Inc., https://www.giagenbioinformatics.com/products/inqenuitypathway-analysis.
37. Modena B D, Kurian S M, Gaber L W, Waalen J, Su A I, Gelbart T, Mondala T S, et al. Gene Expression in Biopsies of Acute Rejection and Interstitial Fibrosis/Tubular Atrophy Reveals Highly Shared Mechanisms That Correlate With Worse Long-Term Outcomes. Am J Transplant 2016; 16:1982-1998.
38. Mengel M, Sis B, Kim D, Chang J, Famulski K S, Hidalgo L G, Einecke G, et al. The molecular phenotype of heart transplant biopsies: relationship to histopathological and clinical variables. Am J Transplant 2010; 10:2105-2115.
39. Sellares J, Reeve J, Loupy A, Mengel M, Sis B, Skene A, de Freitas D G, et al. Molecular diagnosis of antibody-mediated rejection in human kidney transplants. Am J Transplant 2013; 13:971-983.
40. Li L, Khatri P, Sigdel T K, Tran T, Ying L, Vitalone M J, Chen A, et al. A peripheral blood diagnostic test for acute rejection in renal transplantation. Am J Transplant 2012; 12:2710-2718.
41. Suthanthiran M, Schwartz J E, Ding R, Abecassis M, Dadhania D, Samstein B, Knechtle S J, et al. Urinary-cell mRNA profile and acute cellular rejection in kidney allografts. N Engl J Med 2013; 369:20-31.
42. Muthukumar T, Dadhania D, Ding R, Snopkowski C, Naqvi R, Lee J B, Hartono C, et al. Messenger RNA for FOXP3 in the urine of renal-allograft recipients. N Engl J Med 2005; 353:2342-2351.
43. Li B, Hartono C, Ding R, Sharma V K, Ramaswamy R, Qian B, Serur D, et al. Noninvasive diagnosis of renal-allograft rejection by measurement of messenger RNA for perforin and granzyme B in urine. N Engl J Med 2001; 344:947-954.
44. Pham M X, Teuteberg J J, Kfoury A G, Starling R C, Deng M C, Cappola T P, Kao A, et al. Gene-expression profiling for rejection surveillance after cardiac transplantation. N Engl J Med 2010; 362:1890-1900.
45. Rush D N, Gibson I W. Subclinical Inflammation in Renal Transplantation. Transplantation 2019.
46. Kamei H, Masuda S, Nakamura T, Fujimoto Y, Oike F, Ogura Y, Takada Y, et al. Impact of glutathione S-transferase T1 gene polymorphisms on acute cellular rejection in living donor liver transplantation. Transpl Immunol 2013; 28:14-17.
47. Evans P C, Smith S, Hirschfield G, Rigopoulou E, Wreghitt T G, Wight D G, Taylor C J, et al. Recipient HLA-DR3, tumour necrosis factor-alpha promoter allele-2 (tumour necrosis factor-2) and cytomegalovirus infection are interrelated risk factors for chronic rejection of liver grafts. J Hepatol 2001; 34:711-715.
48. Hanvesakul R, Spencer N, Cook M, Gunson B, Hathaway M, Brown R, Nightingale P, et al. Donor HLA-C genotype has a profound impact on the clinical outcome following liver transplantation. Am J Transplant 2008; 8:1931-1941.
49. Toby T, Abecassis M, Kim K, Thomas P, Fellers R, LeDuc R, Kelleher N, et al. Proteoforms in Peripheral Blood Mononuclear Cell as Novel Rejection Biomarkers in Liver Transplant Recipients. Am J Transplant 2017; Accepted 5.6.17 (in press).
50. Kurian S M, Whisenant T, Mas V, Heilman R, Abecassis M, Salomon D R, Moss A, et al. Biomarker Guidelines for High-Dimensional Genomic Studies in Transplantation: Adding Method to the Madness. Transplantation 2017; 101:457-463.
51. Bohne F, Martinez-Llordella M, Lozano J J, Miguel R, Benitez C, Londono M C, Manzia T M, et al. Intra-graft expression of genes involved in iron homeostasis predicts the development of operational tolerance in human liver transplantation. J Clin Invest 2012; 122:368-382.

What is claimed is:

1. A method of adjusting or initiating immunosuppressive therapy in a subject who has undergone a liver transplant, comprising:
a) determining an expression level of one or more genes listed in Table 3 using nucleic acid obtained from the subject, wherein the one or more genes comprises NHS actin remodeling regulator (NHS), endothelial PAS domain protein 1 (EPAS1), complement component 4 binding protein alpha (C4BPA), lymphocyte cytosolic protein 2 (LCP2), deleted in lymphocytic leukemia 2 (DLEU2), NHS like 1 (NHSL1), required for meiotic nuclear division 5 homolog A (RMND5A), lymphocyte activating 3 (LAG3), chitinase 3 like 2 (CHI3L2), gelsolin (GSN), ASAP1 intronic transcript 2 (ASAP1-IT2), NLR family apoptosis inhibitory protein (NAIP), geminin DNA replication inhibitor (GMNN), C-type lectin domain family 4 member E (CLEC4E), X inactive specific transcript (XIST), long intergenic non-protein coding RNA 877 (LINC00877), A-kinase anchoring protein 12 (AKAP12), ATPase family AAA domain containing 2 (ATAD2), or a combination of any of the foregoing;
b) applying the expression level of the genes determined in step (a) to a trained algorithm to classify the subject as (i) undergoing acute rejection (AR) or at risk of undergoing AR or (ii) not undergoing AR or not at risk of undergoing AR; and
c) adjusting or initiating the immunosuppressive therapy in the subject based on classification of the subject in step (b).

2. The method of claim 1, wherein step (a) further comprises determining an expression level of stathmin 1 (STMN1), protein phosphatase 1 regulatory subunit 12B (PPP1R12B), mesoderm specific transcript (MEST), ribonucleotide reductase regulatory subunit M2 (RRM2), senataxin (SETX), thymidylate synthetase (TYMS), GATA binding protein 2 (GATA2), KIAA1324, or a combination of any of the foregoing.

3. The method of claim 1 or claim 2, wherein step (a) comprises determining the expression level of 5, 10, 15, 20, 25, 30, 35, or each of the genes listed in Table 3.

4. A method of adjusting or initiating immunosuppressive therapy in a subject who has undergone a liver transplant, comprising:
a) determining an expression level of each of the genes listed in Table 3 using nucleic acid obtained from the subject;
b) applying the expression level of the genes determined in step (a) to a trained algorithm to classify the subject as (i) undergoing acute rejection (AR) or at risk of undergoing AR or (ii) not undergoing AR or not at risk of undergoing AR; and
c) adjusting or initiating the immunosuppressive therapy in the subject based on classification of the subject in step (b).

5. The method of any one of claims 1-4, wherein the expression level is determined by hybridization to an array or RNA sequencing.

6. The method of any one of claims 1-5, wherein the subject has normal liver function at the time the subject is classified.

7. The method of claim 6, wherein the normal liver function is determined by a liver function test (LFT) in which total bilirubin (TB) is less than 1.5 mg/dL, direct bilirubin is less than 0.5 mg/dL, alkaline phosphatase (AP) is less than 200 U/L, and alanine transaminase (ALT) is less than 60 U/L (males) or less than 36 U/L (females).

8. The method of any one of claims 1-7, further comprising comparing the expression level of each of the genes to a reference expression level that is associated with presence or absence of acute rejection.

9. The method of any one of claims 1-8, wherein the classifying of the subject has a negative predictive value of at least 75%, at least 80%, at least 85%, at least 90%, at least 95%, or at least 99%.

10. The method of any one of claims 1-9, wherein the immunosuppressive therapy or adjusted immunosuppressive therapy comprises administering a calcineurin inhibitor, a mycophenolic acid derivative, an mTOR inhibitor, prednisone, azathioprine, or a combination thereof.

11. The method of any one of claims 1-10, wherein the trained algorithm comprises a random-forest-based algorithm.

12. The method of any one of claims 1-11, wherein the adjusting or initiation of the immunosuppressive therapy comprises increasing dose or frequency of the immunosuppressive therapy in the subject undergoing or at risk of undergoing AR.

13. The method of any one of claims 1-12, wherein the subject is not receiving immunosuppressive therapy at the time the subject is classified.

14. The method of any one of claims 1-11, wherein the adjusting of the immunosuppressive therapy comprises decreasing dose or frequency of the immunosuppressive therapy in the subject not undergoing or not at risk of undergoing AR.

15. The method of any one of claims 1-14, wherein the nucleic acid is obtained from a peripheral blood sample or a biopsy sample.

16. The method of any one of claims 1-15, wherein application of the algorithm in step (b) generates a performance threshold.

17. The method of claim 16, wherein the performance threshold is 0.40, 0.45, 0.50, 0.55, 0.60, 0.65, 0.7, 0.75, or higher.

18. A method of prognosing or diagnosing a liver transplant rejection in a subject, comprising:
(a) obtaining a sample comprising nucleic acid from the subject;
(b) analyzing the nucleic acid to measure expression level of one or more genes listed in Table 3 using nucleic acid obtained from the subject, wherein the one or more genes comprises NHS actin remodeling regulator (NHS), endothelial PAS domain protein 1 (EPAS1), complement component 4 binding protein alpha (C4BPA), lymphocyte cytosolic protein 2 (LCP2), deleted in lymphocytic leukemia 2 (DLEU2), NHS like 1 (NHSL1), required for meiotic nuclear division 5 homolog A (RMND5A), lymphocyte activating 3 (LAG3), chitinase 3 like 2 (CHI3L2), gelsolin (GSN), ASAP1 intronic transcript 2 (ASAP1-IT2), NLR family apoptosis inhibitory protein (NAIP), geminin DNA replication inhibitor (GMNN), C-type lectin domain family 4 member E (CLEC4E), X inactive specific transcript (XIST), long intergenic non-protein coding RNA 877 (LINC00877), A-kinase anchoring protein 12 (AKAP12), ATPase family AAA domain containing 2 (ATAD2), or a combination of any of the foregoing;
(c) prognosing or diagnosing the liver transplant rejection in the subject from the expression levels measured in step (b); and
(d) treating the subject prognosed or diagnosed with liver transplant rejection with immunosuppressive therapy.

19. The method of claim 18, wherein step (a) further comprises determining an expression level of stathmin 1 (STMN1), protein phosphatase 1 regulatory subunit 12B (PPP1R12B), mesoderm specific transcript (MEST), ribonucleotide reductase regulatory subunit M2 (RRM2), senataxin (SETX), thymidylate synthetase (TYMS), GATA binding protein 2 (GATA2), KIAA1324, or a combination of any of the foregoing.

20. The method of claim 18 or claim 19, wherein step (a) comprises determining the expression level of 5, 10, 15, 20, 25, 30, 35, or each of the genes listed in Table 3.

21. A method of prognosing or diagnosing a liver transplant rejection in a subject, comprising:
(a) obtaining a sample comprising nucleic acid from the subject;
(b) analyzing the nucleic acid to measure expression level of each of the genes listed in Table 3;
(c) prognosing or diagnosing the liver transplant rejection in the subject from the expression levels measured in step (b); and
(d) treating the subject prognosed or diagnosed with liver transplant rejection with immunosuppressive therapy.

22. The method of any one of claims 18-21, wherein the nucleic acid is mRNA.

23. The method of claim 22, wherein the mRNA is used to generate complementary DNA (cDNA) prior to step (b).

24. The method of any one of claims 18-23, further comprising contacting the nucleic acid or cDNA with probes, wherein the probes are specific for the one or more genes listed in Table 3.

25. The method of any one of claims 18-24, wherein the sample is a blood sample.

26. The method of any one of claims 18-25, wherein the subject has acute rejection (AR), acute dysfunction no rejection (ADNR), or well-functioning normal transplant (TX).

27. The method of any one of claims 18-26, wherein for each of the genes listed in Table 3, step (c) comprises comparing the expression level of the genes in the subject to one or more reference expression levels of the gene associated with AR, ADNR, or TX.

28. The method of claim 27, wherein step (c) further comprises for each of the genes listed in Table 3 assigning the expression level a value or other designation providing an indication whether the subject has AR, ADNR, or TX.

29. The method of claim 28, wherein the expression level of each of the genes listed in Table 3 is assigned a value on a normalized scale of values associated with a range of expression levels in liver transplant patients with AR, ADNR, or TX.

30. The method of claim 29, wherein the expression level of each of the genes listed in Table 3 is assigned a value or other designation providing an indication that the subject has or is at risk of AR, ADNR, or has well-functioning normal transplant.

31. The method of claim 28, wherein step (c) further comprises combining the values or designations for each of the genes to provide a combined value or designation providing an indication whether the subject has or is at risk of AR, ADNR, or has TX.

32. The method of claim 31, wherein multiple samples are obtained from the subject over time.

33. The method of claim 32, wherein the multiple samples are obtained from the subject at one or more of week 1, week 2, month 1, month 2, month 3, month 6, month 9, month 12, month 15, month 18, month 21, and month 24 after a first sample is obtained.

34. The method of claim 31, wherein the subject is receiving a drug, and a change in the combined value or designation over time provides an indication of the effectiveness of the drug.

35. The method of any one of claims 18-34, wherein the subject has undergone a liver transplant within 1 month, 3 months, 1 year, 2 years, 3 years or 5 years of performing step (a).

36. The method of any one of claims 18-35, wherein step (d) represents a change in treatment and is based on the prognosing or diagnosing.

37. The method of any one of claims 18-36, wherein the subject has received a drug before performing the methods, and the change in treatment comprises administering a higher dose of the drug, administering a lower dose of the drug, stopping administration of the drug, administering an alternative drug, or administering an additional drug.

38. The method of any one of claims 18-37, wherein step (c) is performed by a computer.

39. The method of any one of claims 18-38, wherein the expression levels are measured by quantitative PCR, hybridization to an array, or RNA sequencing.

40. The method of any one of claims 18-39, wherein the subject has normal liver function at the time the subject is prognosed or diagnosed with AR or ADNR.

41. The method of claim 40, wherein the normal liver function is determined by a liver function test (LFT) in which total bilirubin (TB) is less than 1.5 mg/dL, direct bilirubin is less than 0.5 mg/dL, alkaline phosphatase (AP) is less than 200 U/L, and alanine transaminase (ALT) is less than 60 U/L (males) or less than 36 U/L (females).
